(19) United States
(12) Patent Application Publication
(10) Pub. No.: US 2023/0285568 A1
(43) Pub. Date: Sep. 14, 2023

(54) ENGINEERING DIVERSE ANTIGEN-PRESENTING CELLS TO CONTROL ANTIGEN-SPECIFIC RESPONSES

(71) Applicant: University of Maryland, Baltimore, Baltimore, MD (US)

(72) Inventors: Ryan M. PEARSON, Baltimore, MD (US); Gregory SZETO, Seattle, WA (US); Michael ZHANG, Baltimore, MD (US)

(73) Assignee: University of Maryland, Balltimore County, Baltimore, MD (US)

(21) Appl. No.: 18/040,439

(22) PCT Filed: Aug. 5, 2021
(86) PCT No.: PCT/US2021/071112

§ 371 Date: Feb. 3, 2023

Related U.S. Application Data
(60) Provisional application No. 63/061,376, filed on Aug. 5, 2020.

(51) Int. Cl. A61K 47/54; A61K 47/69

(52) U.S. Cl. CPC A61K 47/543; A61K 47/6929; A61K 47/542

(57) ABSTRACT

Phospholipid-conjugated Ags were used as an agnostic delivery platform to cell type, activation state, and inherent uptake capabilities for engineering APCs to control Ag-specific cellular immune responses. Lipid-mediated delivery (termed depoting) of MHC class I and II-restricted Ags successfully loaded resting polyclonal B cells, CD40− activated B cells, and DCs in a dose-dependent manner for priming Ag-specific CD8+ and CD4+ T cells, respectively. When lipid-conjugated Ags were paired with polymer-conjugated Ags and incorporated in nanoparticles (NPs), diverse APCs with varying NP internalization capabilities all processed the lipid-conjugated Ags via depoting while only DCs processed the PLGA-conjugated Ags via endocytosis. Multivariate analyses of cytokine secretions indicated that lipid-conjugated Ags could be distinctly classified from polymer-conjugated Ags. Lipid and PLGA carriers can be rationally paired with Ag combinations to leverage two distinct delivery systems that access multiple Ag processing pathways in diverse APCs, offering a modular delivery platform for engineering ASITs.

### CROSS-REFERENCE TO RELATED APPLICATIONS

This application is a National Stage entry of and claims priority to PCT/US 2021/071112, filed Aug. 5, 2021, which claims the benefit of U.S. Provisional Application Ser. No. 63/061,376, filed 5 Aug. 2020. The entire contents of these two applications hereby are incorporated by reference as if fully set forth herein.

### STATEMENT OF GOVERNMENTAL INTEREST

This invention was made with government support under Grant Number TR003098 awarded by the National Institutes of Health. The government has certain rights in the invention.

### REFERENCE TO SEQUENCE LISTING SUBMITTED VIA EFS-WEB

This application includes an electronically submitted sequence listing in .txt format. The .txt file contains a sequence listing entitled “15024-366PC0_ST25.txt” created on Jan. 22, 2022, and is 1,499 bytes in size. The sequence listing contained in this .txt file is part of the specification and is hereby incorporated by reference herein in its entirety.

### BACKGROUND

### 1. Field of the Invention

This invention relates to the field of medicine and in particular to the field of vaccine production, and in particular of APC-targeted immunotherapies to generate antigen-specific T cell responses for application in cancer immunity, immune tolerance induction for application in autoimmune diseases such as multiple sclerosis, and immune tolerance for application in allergies from food and environmental sources.

### 2. Background of the Invention

Despite improved diagnostics and therapeutics in the clinic, cancer continues to be one of the leading causes of death. In 2018, an estimated 18 million new cancer cases emerged in the world, with about 9.6 million resulting in death. Traditional therapies for combating cancer include chemotherapy, radiotherapy, and invasive surgeries. However, many aggressive cancers are either refractory to or can rebound from these treatments and require further intervention to halt disease progression.

The immune system plays a vital role in the defense against pathogens and other chronic diseases. The elimination of cancer cells is broadly attributed to activated CD4⁺ and cytotoxic CD8⁺ T cells. Dysfunctional anti-tumor T cells can result in tumor progression and poor prognosis Immunotherapy can resuscitate the dysfunctional immune response and enable effective clinical treatments against established cancers. However, major challenges in cancer immunotherapy including the lack of approaches which enable the specific delivery of therapeutic cargo to target cells in vivo, the relatively low abundance of antigen (Ag) presenting cells (APCs) available to induce anti-tumor adaptive immune responses, and the need to incorporate adjuvants to induce immunogenic Ag-specific T cell stimulation still remain Therefore, there is a need in the art for methods to systematically address these limitations using nanotechnology and immune engineering to improve cancer vaccine efficacy.

In addition, another major challenge in immunotherapy has been enabling the delivery of therapeutic cargo to specifically and effectively mitigate aberrant immune activation in diseases such as multiple sclerosis (MS). Conventional therapies for MS are not antigen (Ag)-specific and primarily focus on addressing disease symptoms through anti-inflammatory or immunosuppressive mechanisms such as targeting the T cell receptor, co-signaling molecules, cytokines, or others. Technologies that induce Ag-specific immune tolerance without the need for co-administration of immunomodulators offer significant potential to improve the treatment of autoimmune diseases and to mitigate the numerous side effects associated with non-specific immunomodulation.

Vaccine research has traditionally focused on adjuvants and soluble antigenic (Ag) peptides or proteins to instruct Ag-specific immune responses. Ag dose, trafficking, and cell type are key variables known to affect vaccine efficacy. However, no current technologies are available which offer the ability to simultaneously and precisely control each of these critical aspects necessary to initiate Ag-specific anti-cancer immune responses. APCs play a critical role in the generation of T cell-mediated immune responses. B cells are an underappreciated APC for therapeutic cancer vaccines, however their relatively high abundance in the peripheral blood, enhanced in vivo trafficking to lymphoid sites, and higher proliferative potential compared to dendritic cells (DCs) make them an ideal candidate for in vivo immunomodulation.

Antigen-specific immunotherapies (ASITs) have made tremendous strides in modern medicine by training the host immune system to recognize antigens (Ags) through cellular immune responses. Successful ASITs deliver Ags to Ag-presenting cells (APCs) in vivo or ex vivo, which then train the adaptive immune response such as T cells to provide Ag-specific immunity or tolerance. Dendritic cells (DCs) are professional APCs and therapeutically used for efficient non-specific Ag uptake, processing, and MHC class II- and class I-restricted presentation to prime CD4⁺ and CD8⁺ T cells, respectively. While DCs are actively investigated in ASIT, they are hampered by the potent but variable Ag-processing capabilities among different DC subsets or DC activation states, resulting in insufficient and inconsistent Ag delivery in vivo for priming desired CD4⁺ or CD8⁺ T cells. Cell-based immunotherapy strategies deliver Ags to harvested DCs to avoid challenges associated with in vivo delivery, but the scarcity of DCs in blood and poor DC expansion ex vivo hinder scalable manufacturing for clinical infusions. The lack of FDA-approved ASITs suggests that innovative strategies are required to simply and reliably deliver Ags to diverse APC candidates for mounting effective cellular immunity or tolerance.

Alternative APCs can be harnessed for ASITs and some have advantages with respect to manufacturing over DCs. B cells in particular are more abundant in the peripheral blood, easier to expand into large numbers compared to DCs ex vivo, and can be maintained for weeks ex vivo as a supply source for clinical infusions. These attributes are advantageous for engineering cell-based immunotherapies, where large quantities of APCs are required to satisfy the manufacturing demands for even one patient infusion.

However, B cells are inefficient at non-specific Ag uptake and Ag cross-presentation when compared to DCs, limiting therapeutic versatility in activating cellular immune responses. B cells can instead leverage Ag-specific B-cell receptors (BCRs) to endocytose Ags and prime CD4⁺ T cells, a mechanism that is largely linked to B-cell activation state. Dose-dependent Ag presentation and APC activation signaling have each demonstrated control over the magnitude and phenotype of T-cell activation. The mechanistic dependence of Ag uptake on cellular activation constrains broad utility of B cells and other cells as therapeutic APCs for selectively inducing potent T-cell immunity or tolerance. Thus, there is a need in the art for strategies for delivering Ags to APCs without dependence on cellular activation signals in order to overcome Ag delivery challenges that currently hinders all APCs.

### SUMMARY OF THE INVENTION

Successful engineering strategies to overcome the functional limitations of using B cells as APCs for ASIT should be able to control Ag dosing, promote sufficient Ag presentation, induce Ag-specific CD4⁺ and CD8⁺ T cells, and produce phenotypic profiles appropriate to the particular disease indications. Currently available methods to promote Ag cross-presentation to activate CD8⁺ T cells include direct Ag delivery into B-cell cytosols by electroporation, transfection, viral vector-based transduction, or mechanical perturbations using microfluidic systems. However, these methods rely on plasma membrane disruptions that can be difficult to consistently control or to scale-up for large B-cell batch sizes in clinical settings, and require cell activation. Without these factors engineering the cells can lead to irreversibly compromised membrane integrity and decreased cell viability.

This technology enables the design and implementation of a broader landscape of APC-targeted immunotherapies and enhance the use of non-traditional APCs such as B cells in experimental and therapeutic scenarios to generate Ag-specific T cell responses for applications in cancer immunotherapy or immune tolerance induction. This enables an approach that has not been available previously. LAg-nanos are not patient-specific and offer numerous advantageous over cell-based therapies including lower cost, lower variability, greater control over physicochemical properties, greater shelf-life, easier handling, and better scalability.

The most potent APCs for antigen (Ag) uptake and activating cellular immunity or tolerance are dendritic cells (DCs). Despite their development for ASIT, DCs face several challenges that hinder therapeutic translation, including reliable dosing of Ag for tunable immune response, low abundance in peripheral blood for clinical targeting, complex conditioning regimens for maintaining differentiation ex vivo, and poor proliferation potential. B cells are an alternative APC source due to their higher abundance in the peripheral blood and proliferation potential compared to DCs. While B cells have potential as therapeutic APCs for eliciting cellular immunity and tolerance, their overall poor Ag uptake capabilities compromise controllable priming of CD4⁺ and CD8⁺ T cells.

Here, phospholipid-conjugated Ags were used as a delivery strategy agonistic to cell type, activation state, and inherent uptake capabilities for engineering APCs to control Ag-specific cellular immune responses. Lipid-mediated delivery (depoting) of MHC class I and II-restricted Ags successfully loaded resting polyclonal B cells, CD40-activated B cells, and DCs in a dose-dependent manner for priming Ag-specific CD8⁺ and CD4⁺ T cells, respectively. When lipid-conjugated Ags were paired with polymer-conjugated Ags and incorporated in nanoparticles (NPs), diverse APCs with varying NP internalization capabilities all processed the lipid-conjugated Ags via depoting while only DCs processed the PLGA-conjugated Ags via endocytosis. Multivariate analyses of cytokine secretions indicated that lipid-conjugated Ags could be distinctly classified from polymer-conjugated Ags. Altogether, we demonstrated that lipid and PLGA carriers can be rationally paired with Ag combinations to leverage two distinct delivery systems that access multiple Ag processing pathways in diverse APCs, offering a modular delivery platform for engineering ASITs.

Specifically, the invention relates to a method of presenting antigen in a cell, comprising:
(a) obtaining a peptide or protein antigen;
(b) covalently linking the antigen to a lipid to form a conjugate;
(c) introducing the lipid-antigen conjugate into a nanoparticle; and
(d) loading the cells with antigen by contacting the cells with the nanoparticles, wherein the lipid is compatible with incorporation into nanoparticles and delivering the antigen into the plasma membrane of cells, wherein the nanoparticle comprises biomaterials compatible with the lipid and the cell and can deliver the antigen to the cell, and wherein the antigen-loaded cells produce an antigen-specific T cell response.

In certain embodiments of the invention, the cells are macrophages, dendritic cells, resting B cells, or activated B cells. The antigen can be an MHCI antigen or an MHCII antigen. In some embodiments, the lipid is selected from the group consisting of 1,2-distearoyl-sn-glycero-3-phosphorylethanolamine (DSPE). DSPE-PEG, stearic acid, stearic acid-PEG, cholesterol, cholesterol-PEG, distearoylphosphatidylcholine (DSPC), and DSPC-PEG. In some embodiments, the nanoparticle comprises poly(lactic-co-glycolic acid), poly(lactic acid), poly(ε-caprolactone), polystyrene, poly(methyl methacrylate), N-(2-hydroxypropyl)-methacrylamide, poly(ethylene glycol), poly(glycolic acid), polyanhydrides, poly(cyano-acrylates), poly(maleic acid), poly(N-vinyl pyrrolidine), chitosan, hyaluronic acid, albumin, heparin, DSPE, DSPE-PEG, palmitoyl, stearic acid, stearic acid-PEG, cholesterol, cholesterol-PEG, DSPC, DSPC-PEG, or mixtures thereof, preferably poly(lactic-co-glycolic acid), poly(lactic acid), or a mixture thereof.

In certain embodiments, the antigen-specific T cell response is immune tolerance; in others, the antigen-specific T cell response is immune stimulation.

The antigen loading can be performed ex vivo or in vivo.

In particular embodiments, the invention incudes an antigen presenting B cell produced by the method described above.

Certain embodiments of the invention include a nanoparticle comprising an antigen-lipid conjugate, wherein the antigen is a protein or peptide, and wherein the lipid is compatible with incorporation into nanoparticles and delivering the antigen into the plasma membrane of cells, and wherein the nanoparticle comprises poly(lactic-co-glycolic acid), poly(lactic acid), poly(ε-caprolactone), polystyrene, poly(methyl methacrylate), N-(2-hydroxypropyl)-methacrylamide, poly(ethylene glycol), poly(glycolic acid), polyanhydrides, poly(cyano-acrylates), poly(maleic acid), poly(N-vinyl pyrrolidine), chitosan, hyaluronic acid, albumin, heparin, DSPE, DSPE-PEG, stearic acid, stearic acid-PEG, cholesterol, cholesterol-PEG, DSPC, DSPC-PEG, palmitoyl, or mixtures thereof, preferably poly(lactic-co-glycolic acid), poly(lactic acid), or a mixture thereof.

In other embodiments, the invention comprises an antigen presenting cell which has been loaded with antigen by interaction with the nanoparticle described above.

The invention also comprises, in certain embodiments, a method of presenting antigen in a resting B cell or an activated B cell comprising contacting the resting B cell or activated B cell with the nanoparticle of claim 12.

In some embodiments, the invention comprises a method of treating autoimmune disease or cancer in a subject in need comprising administering an antigen presenting cell as described herein, or a method of inducing an antigen-specific immune reaction in a subject in need thereof, comprising administering the antigen presenting cell as described herein to the subject. In preferred embodiments, the subject is suffering from cancer or an autoimmune disorder such as multiple sclerosis.

### BRIEF SUMMARY OF THE DRAWINGS

FIG. 1 is a schematic drawing of LAg-nano as a novel immune-modulating platform for the induction of Ag-specific immune responses in CD4⁺ and CD8⁺ T cells. The schematic shows the LAg-nano formulation and a representation of the 2 types of cargo delivery mechanisms enabled.

FIG. 2 is a schematic showing various combinations of Polymer-Ag, Lipid-Ag, polymer, or lipid, combined in a modular, ‘mix-and-match’ approach to formulate a variety of nanoparticles for the delivery of antigenic cargoes.

FIG. 3 is a 1H-NMR spectrum of DSPE-PEG2000-NHS in d6-DMSO.

FIG. 4 is a 1H-NMR spectrum of DSPE-PEG2000-OVA323-339 in d6-DMSO.

FIG. 5 is a 1H-NMR spectrum of DSPE-PEG2000-SIINFEKL (SEQ ID NO:1) in d6-DMSO.

FIG. 6 is a 1H-NMR spectrum of DSPE-PEG2000-Ea52-68 in d6-DMSO.

FIG. 7 is a 1H-NMR spectrum of PLGA-Ea52-68 in d6-DMSO.

FIG. 8 is a 1H-NMR spectrum of PLGA-SIINFEKL (SEQ ID NO:1) in d6-DMSO.

FIG. 9A through FIG. 9C shows that lipo-Ag conjugates load in a time- and concentration-dependent manner FIG. 9A is a graph of relative density of lipo-GP100 loading on CD11c⁺ mouse bone marrow-derived dendritic cells (BMDCs) and B220⁺ B cells. The data shown are a mean ±s.d (n=3-4 independent samples). FIG. 9B is a graph of lipo-GP100 loading on B cells, analyzed cells by confocal microscopy (FIG. 9C).

FIG. 10A through FIG. 10C shows that lipo-Ag conjugate enables APC internalization and promotes MHC I-restricted antigen presentation to cognate CD8⁺ T cells. FIG. 10A and FIG. 10B are graphs of lipo-GP100 (20-mer) loaded onto BMDCs and B cells and was functionally presented by APCs to GP100-specific CD8⁺ T cells (PMELs) similar to minimal GP100 (9-mer) presentation, as determined by CFSE dye dilution after 3-day co-culture. Percentages represent percent of divided PMELs. FIG. 10C is a graph of lipo-GP100 internalization and presentation by B cells showing concentration-dependence.

FIG. 11A through FIG. 11H shows that lipo-Ag enables APC internalization and promotes MHC II-restricted presentation to cognate CD4⁺ T cells. BMDCs and B cells loaded with unmodified ovalbumin (OVA) MHC II-restricted antigen (OVA₃₂₃₋₃₃₉) or lipo-OVA₃₂₃₋₃₃₉ were co-cultured with OVA-specific CD4⁺ T (OT-II) cells for 3 days. Representative OT-II cell activation as shown by CFSE dye dilution (FIG. 12A and FIG. 12B) and CD25 expression using flow cytometry (FIG. 12C and FIG. 12D). FIG. 12E, FIG. 12F, FIG. 12G, and FIG. 12G present data on the quantitation of proliferation and division indices of OT-II T cells. Data showed mean ±s.d. n=2 independent samples.

FIG. 12A through FIG. 12F shows lipo-Ag conjugates and LAg- +PAg-nano loaded onto APCs and presented on surface MHCs. Data for B220+ (FIG. 12A and FIG. 12C), F4/80+ (FIG. 12B and FIG. 12E), and CD11c+ (FIG. 12C and FIG. 12F) APCs are presented. The APCs were loaded with SIINFEKL (SEQ ID NO:1) and Ea peptides as lipo-Ag conjugates or L/P-Ag PLGA NPs, (P-Ea+P-SIINFEKL(SEQ ID NO:1))-nano or (P-Ea+L-SIINFEKL (SEQ ID NO:1)-nano. Presentation of SIINFEKL (SEQ ID NO:1) on MHC I and Ea on MHC II was respectively determined by MFI with flow cytometry. P denotes PLGA polymer (PAg-nano), L denotes Lipid (LAg-nano). Data showed mean ±s.d. n=3 replicates.

FIG. 13A through FIG. 13B shows lipo-Ag conjugates and P/L-Ag-nano enable MHC I- and MHC II-restricted presentation by BMDCs for priming cognate CD4⁺ (OT-II; FIG. 13A) and CD8⁺ (OT-I; FIG. 13B) T cells. BMDCs were loaded with OVA-derived Ags as lipo-Ag conjugates, or P/L-Ag blends in NPs, (P-OVA₃₂₃₋₃₃₉+L-SIINFEKL (SEQ ID NO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL (SEQ ID NO:1))-nano, and co-cultured with OT-II (FIG. 13A) or OT-I (FIG. 13B) cells for 3 days. Representative histograms show T-cell proliferation as determined by CFSE dye dilution using flow cytometry. P denotes PLGA polymer (PAg-nano), L denotes Lipid (LAg-nano).

FIG. 14A through FIG. 14B presents data on lipo-Ag conjugates and P/L-Ag-nano enable MHC I- and MHC II-restricted presentation by B cells for priming cognate CD4⁺ (OT-II) and CD8⁺ (OT-I) T cells. FIG. 14A: OT-II and FIG. 14B: OT-I cells for 3 days. Representative histograms show T-cell proliferation as determined by CFSE dye dilution using flow cytometry. P denotes PLGA polymer (PAg-nano); L denotes Lipid (LAg-nano).

FIG. 15A through FIG. 15D (SIINFEKL (SEQ ID NO: 1)) presents data on lipid-based biomaterials enable MHC I- and MHC II-restricted presentation by B cells for priming cognate CD4⁺ (OT-II; FIG. 15A and FIG. 15C) and CD8⁺ (OT-I; FIG. 15B and 15D) T cells for 3 days. T-cell proliferation was determined by CFSE dye dilution using flow cytometry, and quantification of proliferation and division indices was determined. P denotes PLGA polymer (PAg-nano), L denotes Lipid (LAg-nano). Data showed mean ±s.d. n=2-4 independent samples.

FIG. 16A through FIG. 16D (SIINFEKL (SEQ ID NO: 1)) shows data from lipid-based biomaterials enable MHC I- and MHC II-restricted presentation by CD40 B cells for priming cognate OT-II and OT-I T cells. FIG. 16A and FIG. 16C: OT-II and FIG. 16B and FIG. 16D: OT-II cells. Data showed mean ±s.d. n=2 independent samples.

FIG. 17A through FIG. 17D (SIINFEKL (SEQ ID NO: 1)) shows that lipid-based biomaterials enable MHC I- and MHC II-restricted presentation by B and CD40 B cells for priming cognate OT-II (FIG. 17A and FIG. 17B) and OT-I (FIG. 17C and FIG. 17D) T cells.

FIG. 18 (SIINFEKL (SEQ ID NO: 1)) is a heatmap showing that biomaterial conjugates drive differential cytokine secretion profiles. The heatmap represents normalized concentrations (z-scores) of 32 cytokines for each biomaterial-based Ag treatment as measured by Luminex. P denotes PLGA polymer (PAg-nano); L denotes Lipid (LAg-nano). n=2 replicates.

FIG. 19A through FIG. 19I (SIINFEKL (SEQ ID NO: 1))shows that lipo-Ag conjugates and P/L-Ag-nano differentially enable MHC I- and MHC II-restricted presentation by APCs and induce pro-inflammatory cytokine secretion. TNFα (FIG. 19A, FIG. 19B, and FIG. 19C), IL-2 (FIG. 19D, FIG. 19E, and FIG. 19F), and IFNγ (FIG. 19G, FIG. 19H, and FIG. 19I) cytokine levels for each biomaterial-based treatment were measured by Luminex. P denotes PLGA polymer (PAg-nano); L denotes Lipid (LAg-nano). Data showed mean ±s.d. n=2 replicates.

### DETAILED DESCRIPTION OF THE INVENTION

### 1. DEFINITIONS

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

The term “about,” as used herein, means plus or minus 20 percent of the recited value, so that, for example, “about 0.125” means 0.125±0.025, and “about 1.0” means 1.0±0.2.

The term “proliferation index,” as used herein, is defined as the total number of cell divisions divided by the number of divided cells; the term “division index,” as used herein is the total number of cell divisions divided by the number of total original cells.

The term “LAg” or “Lipo-Ag,” as used herein, refers to a lipid-tailed antigen, i.e., a peptide antigen covalently linked to a lipid molecule. For the work described here, the lipid DSPE-PEG2000-NHS (Lipo) was linked to the peptide antigen. The term “PAg” or “Poly-Ag,” as used herein, refer to polymer-antigen.

The term “nanoparticle,” as used herein, refers to a particle from about 10 nm to about 1500 nm in diameter, preferably about 100 nm to about 1200 nm, more preferably about 200 nm to about 1000 nm and most preferably about 400 nm to about 600 nm or about 500 nm. Such particles are composed of any of the following including but not limited to: poly(lactic-co-glycolic acid), poly(lactic acid), poly(ε-caprolactone), polystyrene, poly(methyl methacrylate), N-(2-hydroxypropyl)-methacrylamide, poly(ethylene glycol), poly(glycolic acid), polyanhydrides, polycyanoacrylates, poly(maleic acid), poly(N-vinyl pyrrolidine), chitosan, hyaluronic acid, alginate, albumin, heparin, metals including but not limited to silver, gold, silica, or lipids including but not limited to DSPE-PEG, stearic acid-PEG, cholesterol-PEG, DSPC-PEG, and their non-PEGylated counterparts, or mixtures thereof.

The term “tolerogenic nanoparticles” or “tNP,” as used herein, refers to nanoparticles (typically prepared using poly(lactic-co-glycolic acid)) which contains an antigen or antigens of interest and are capable of inducing an antigen-specific T cell tolerance. The term “immunomodulatory nanoparticles” or “iNP,” as used herein, refers to nanoparticles which are designed with features comprised of biomaterials that have been designed to actively modulate (enhance, skew, or suppress) immune responses.

The term “LAg-nano,” as used herein, refers to nanoparticles that are loaded with LAg.

The term “subject,” as used herein refers to any host or patient, including any mammal. Humans, primates, farm animals and livestock, companion animals, and laboratory animals are included, such as human, apes, monkeys, rats, mice, rabbits, bovines, equines, ovines, caprines, dogs, cats and the like. A “subject in need” refers to any subject that suffers from a condition that can be benefitted by the methods and compositions described herein. Such subjects generally are those which suffer from any autoimmune disease (e.g., multiple sclerosis, type 1 diabetes, Graves' disease, psoriasis, inflammatory bowel disease, celiac disease, rheumatoid arthritis, systemic lupus erythematosus, graft vs. host disease/transplant rejection, ankylosing spondylitis, Sjogren's syndrome, and the like), any condition arising from an abnormal or abnormally strong immune response (e.g., seasonal allergies, environmental allergies, food allergies, and the like), any benign or malignant hyperproliferative disorder (e.g., cancer, benign tumor, and the like), infection (e.g., viral or bacterial), or a subject in need of a vaccine.

The term “antigen presenting cell” (APC), as used herein, refers to a cell that displays antigen, bound by major histocompatibility complex (MHC) proteins on its surface. APC process antigens and present them to T cells which can be stimulated or tolerized by this interaction. Antigen presenting cells include macrophages, B cells, and dendritic cells (DC), but also can include any nucleated cells in the body, generally mast cells, neutrophils, endothelial cells and epithelial cells.

The term “B cell,” as used herein, refers to a subtype of lymphocyte which is activated by antigen and operates as an antigen presenting cells. A “resting” B cell B cell is one that has not been stimulated by any agent (such as anti-CD40, TLR ligands, anti-B cell receptor antibodies, and the like). An “activated B cell” is one which has been activated by one or more of the indicated agents.

The term “lipid,” as used herein, refers to an of the common lipids known in the art, including cholesterol, fatty acids, triglycerides, phospholipids, and the like. Those most useful for the invention described here are phospholipids.

The term “loading,” as used herein, refers to incorporation of the antigen or lipid-antigen into nanoparticles.

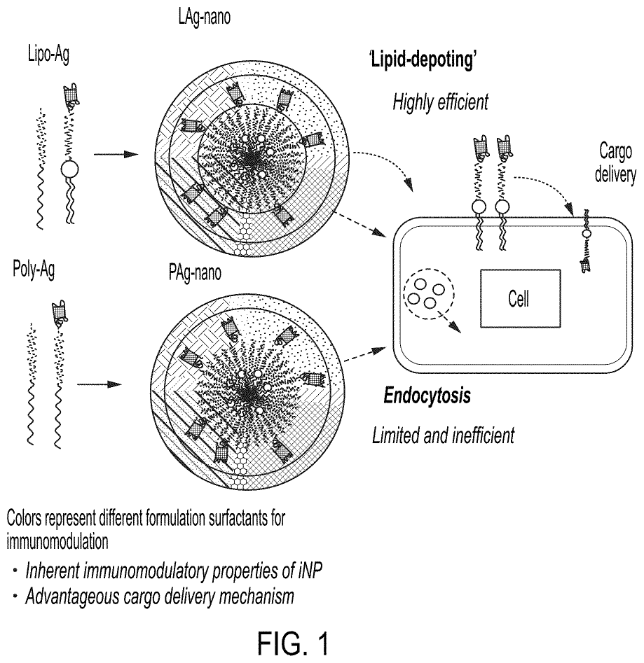

The term “depoting,” as used herein, refers to a lipid-mediated mechanism which mimics the natural insertion of glycosylphosphatidylinositol (GPI)-anchored proteins into cells or membranes. See FIG. 1.

### 2. OVERVIEW

The approach used here combines the advantages of two distinct Ag delivery technologies (Lipo-Ag membrane insertion and iNPs) to overcome limitations associated with Ag delivery to B cells (a unique approach that enables enhanced accessibility of immune cells with limited phagocytic potential) allows for the controllable Ag delivery (quantity and release rate), and allows for immune responses to be engineered into a single versatile delivery platform. This work expands upon and improves current knowledge of cellular vaccines in three ways: 1) establishing a modular, lipid-mediated delivery platform for diverse Ag to B cells and other APCs, 2) probing B cell biology in the context of intracellular processing and presentation of peptide epitopes on MHC I and MHC II, and 3) B cells as an effective APC option for antigen-specific immunotherapy.

Using lipid association with plasma membranes and delivery using iNPs, the invention provides a modular system for delivering multiple and diverse cargo to diverse cells, expanding the possibilities of cargo delivery beyond APCs to potentially any cell type. As an in vivo-application, the APC-targeted therapeutic vaccine system is positioned for high translational potential. Specifically, this project highlights the subcellular compartments to which vaccine cargo can be delivered in B cells that may otherwise not be easily possible with natural physiological B cell biology. Successful integration of this delivery system with multiple APCs can be validated in mouse immune disease models in protective and therapeutic settings, demonstrating not only the utility of this engineered strategy for cancer vaccine research, but also for other infectious and chronic illnesses.

Nanoparticle (NP) carriers have been extensively developed for carrying diverse peptide and protein Ags with decreased cellular toxicities. NPs comprised of lipids, poly(lactic-co-glycolic acid) (PLGA) and other polymers, or hybrid lipid-PLGA are sequestered to APCs in vivo for increased Ag uptake, MHC-restricted presentation, and Ag-specific T-cell activation. DCs can phagocytose and endocytose NPs through active uptake mechanisms, but B cells cannot, hindering the therapeutic potential of many NP-based delivery platforms that aim to harness B cells as APCs.

Lipid-based bioconjugates can promote delivery of therapeutic cargoes to APCs by inserting into cell plasma membranes for internalization, circumventing active uptake mechanisms for accessing intracellular pathways by mimicking the natural insertion of glycosylphosphatidylinositol (GPI)-anchored proteins via a lipid-mediated mechanism termed “depoting.” Lipid conjugation to adjuvants enabled precise control and enhanced delivery to B and T cells, activating multiple autocrine or paracrine immune signaling pathways for boosting immune responses. Extending that strategy for precise Ag delivery to access MHC I and MHC II-restricted Ag presentation pathways in diverse APCs offers significant potential for use in broad ASIT.

### 3. SUMMARY OF RESULTS

This invention provides nanoparticles prepared using biomaterial-Ag conjugates and formulated into LAg-nano or PAg-nano. These can serve as an efficient delivery system of peptides and whole proteins to an expanded set of APCs to offer enhanced T cell activation and response for fine-tuning therapeutic immune responses. This approach can uncover the differential mechanisms by which APCs process and present Ags in order to provide strategies for rational design of Ag and adjuvant delivery to enhance the efficacy of in vivo-applied vaccines and immune tolerizing biomaterials targeted towards a wide variety of APCs.

Here, we describe the development and evaluation of lipid-based Ag delivery systems to expand the accessibility of APCs available for ASIT to B cells. We conjugated exact MHC I- or MHC II-restricted peptide Ags to lipids (L-Ag) to biomimetically insert into plasma membranes of DCs and B cells for activating CD8⁺ and CD4⁺ T cells. We hypothesized that the incorporating L-Ag and PLGA-conjugated Ag (P-Ag) into PLGA NPs, forming lipid-polymer hybrid NPs, could take advantage of two distinct mechanisms of uptake: (1) phagocytosis/endocytosis (provided by the NP) and (2) “depoting” mediated by L-Ag.

Using a series of systematic co-culture experiments, the APC-dependent efficiency of induction of Ag-specific CD4⁺ and CD8⁺ T cell activation, proliferation, and cytokine secretions were examined as a function of L-Ag or P-Ag mediated PLGA NP delivery to DCs, non-activated B cells (B-APCs), and CD40 pathway-activated B cells (CD40 B-APCs). This study demonstrates “depoting” using L-Ag PLGA NPs as a novel Ag delivery mechanism, which combines the advantages of lipid- and PLGA-mediated delivery systems to engineer diverse APCs to and prime CD4⁺ and CD8⁺ T cells that were traditionally inaccessible to NP Ag delivery platforms. These findings have broad applications for use in applications including cancer immunotherapy, vaccine development, and autoimmunity.

### 4. EMBODIMENTS OF THE INVENTION

The technological advancements that underlie this invention stem from the combination of two distinct technologies: (1) lipid-tailed Ags (Lipo-Ag) and (2) tolerogenic nanoparticles (tNP). LAg-nano, Lipo-Ag-containing tNPs, are a highly controlled and modular particle-based platform capable of inducing Ag-specific CD4⁺ and CD8⁺ T cell responses using a broad array of APCs, including DCs, macrophages, and most notably, B cells. Lipo-Ags enable efficient, minimally perturbative ex vivo delivery of Ags to various immune cell types and induce potent T cell responses. Furthermore, covalent modification of lipids with Ags enables simple, modular, and stoichiometric incorporation of Lipo-Ag into tNP formulations with reproducible control over the Ag loading and release properties of the particles.

Ag delivery using tNPs have successfully induced Ag-specific immune tolerance in various rodent models of autoimmunity, allergy, and allogenic cell transplantation. Delivery of Ag using LAg-nano according to the invention allows B cells to be used as targetable APCs to induce Ag-specific tolerance or other immune reactions by addressing previous deficiencies in Ag processing and presentation mechanisms of tNP. Therefore, LAg-nanos are a potent, tolerance-inducing technology that programs B cells through non-genetic engineering mechanisms to mitigate aberrant and pathogenic T cell responses, for example in MS.

In MS, B cell depletion with anti-CD20 has been able to eliminate cells responsible for the production of pathogenic autoantibodies. Also, Ag-specific B cells can recognize and internalize Ags through their B cell receptor (BCR), which induces their activation and subsequent differentiation into antibody-secreting plasma cells. LAg-nanos do not rely on the B cell receptor to induce antigen-specific immune responses. This invention takes advantage of the Ag presenting capabilities (i.e. major histocompatibility complex (MHC I and II)) and low levels of co-stimulatory molecule expression in resting B cells to induce tolerance rather than relying on depletion strategies (non-specific) nor BCR targeting to induce Ag-specific adaptive immune responses. Resting B cells are generally regarded as poor APCs because of limited Ag uptake, however the methods of this invention are able to use these plentiful cells to produce immune response induction for tumor treatment or for immune tolerance.

The type of Ag-specific immune response induced is highly dependent on the context of the Ag presenting cell (APC):T cell interaction. Numerous APCs have been implicated in tolerance induction and traditionally, dendritic cells (DCs) and macrophages have been the primary APC subsets targeted for tolerance therapies. Recognizing the relative scarcity of DCs and macrophages in the body (˜1-6% DCs and macrophages or splenocytes), this invention provides a nanoparticle and method which is useful for inducing an immune response, including for tolerance-inducing therapies for the treatment of immune diseases such as multiple sclerosis and as vaccines for treatment of cancer.

In summary, with respect to the invention, B cells are capable of inducing tolerance and are present in much greater proportion (10-100 fold greater) than other professional APCs in the body. The methods control the context of Ag presentation and expand the accessible APC repertoire to highly abundant B cell populations (45-55% of splenocytes).

The Lipo-Ag according to this invention is made up of PEGylated phospholipid conjugated to an Ag (DSPE-PEG2000-Ag). The insertion of Lipo-Ags into membranes is similar to the natural insertion of GPI-anchored proteins in that they can insert into splenocytes and resting lymphocytes (B cells, CD4⁺ T cells, CD8⁺ T cells). However, to achieve selective manipulation of specific immune cells, modifications must occur ex vivo thus requiring time-intensive experimentation and stringent aseptic techniques. Formulation of Lipo-Ags into nanoparticles (LAg-nano) eliminates the need for ex vivo manipulation and enables immune cell selective properties to be engineered into the carrier.

Using polymer-coupled encephalitogenic Ags (Poly-Ag) incorporated into 500 nm poly(lactide-co-glycolide) (PLGA) nanoparticles (PAg-nano) has enabled precise control over Ag loading, near complete elimination of Ag burst release, and induced robust immune tolerance using EAE mouse models induced using single or multiple Ags. Particles distributed into macrophages and DCs in the liver and spleen after intravenous injection.

Delivery of LAg-nanos containing OVA₃₂₃₋₃₃₉ or OVA₂₅₇₋₂₆₄ Lipo-Ags to B cells could induce Ag-specific OTI CD4⁺ and OTII CD8⁺ T cell responses. PAg-nano delivery to B cells was unable to induce a significant T cell response in B cells. However, both LAg-nano and PAg-nano performed similarly in an experiment using DCs. To establish the mechanisms that underlie the effectiveness of LAg-nano, a series of Lipo-Ags and LAg-nanos containing MHC class I (OVA₂₅₇₋₂₆₄) and MHC class II (OVA₃₂₃₋₃₃₉, Ea₅₂₋₆₈) Ags were synthesized and formulated as similarly described for PAg-nanos. Lipo-Ags were incorporated at precise ratios to achieve Ag loadings of MHCI and MHCII peptide Ags in LAg-nanos with either 8 or 25 μg/mg (denoted as Lo or Hi loading).

Uptake of LAg-nanos can be evaluated using fluorescently-labeled Lipo-Ags and compared to Poly-Ag and PAg-nanos using confocal microscopy and flow cytometry. Ag-presentation can be assessed using two methods in resting and CD40-activated B cells, and DCs: i) flow cytometry for presentation of peptide on MHCI (OVA₂₅₇₋₂₆₄) or MHCII (Ea₅₂₋₆₈) presentation of peptide on MHCII; and ii) induced CD4⁺ or CD8⁺ T cell proliferative responses using OTI or OTII T cells. The resulting extent of T cell proliferation can be assessed by flow cytometry. To determine any potential non-specific activation of B cells following culture with Lipo-Ag or LAg-nano, the surface expression of MHCII, CD69, and other co-stimulatory and co-inhibitory molecules can be assessed by flow cytometry. Levels of Th1/17- and Th2-type cytokines can be measured from the culture supernatants using a 30-plex mouse inflammation panel using Luminex LAg-nano formulations which induce strong activation of CD4+ and CD8+ T cells are preferred for cancer applications and diminished activation of CD4+ and CD8+ T cells, as well as high levels of Tregs are preferred for immune tolerance applications.

Ag delivery to polyclonal B cells is distinct from DCs because their non-specific Ag uptake is less frequent and they largely uptake Ag through binding specific proteins by B cell receptor-mediated endocytosis. Thus, the development of technologies that can expand the APC subsets available for immunomodulation in vivo offer significant potential to enhance Ag-specific immunity for cancer eradication. The development of nanoparticles (NPs) for the treatment of cancer holds significant promise to improve efficacy and patient outcomes by affording precise control over the type of immune response generated by tailoring specific features through engineering cellular interactions, Ag delivery, and NP physiochemical properties.

Cell-based vaccines have shown therapeutic potential as immunotherapies for chronic diseases. DCs are commonly used as antigen presenting cells (APCs) to process and present disease-associated antigen (Ag) for priming of T cells. However, the low abundance of DCs in the peripheral blood, complex conditioning and adjuvant regimens for maintaining differentiation, and relatively poor proliferation potential highlights the need for advancements in alternative APCs. Polyclonal B cells have also received attention as potential APCs for cancer immunotherapy due to advantages such as high abundance in peripheral blood, ability to traffic from blood to secondary lymphoid organs, high proliferative capacity, and prolonged lifespan, but this so far has not been shown to be viable.

A major consideration for designing cell-based vaccines is the mechanism of Ag delivery. In this study, Ag processing and presentation in DCs and B cells were compared by engineering lipid- and PLGA-based Ag (LAg-nano and PAg-nano) carriers to control the efficiency and fate of Ag. The development of technologies that can expand the APC subsets available for immunomodulation in vivo offer significant potential to enhance Ag-specific immunity for cancer eradication or tolerance induction.

Peptides were conjugated to the terminal carboxylic acid group of PLGA or DSPE-PEG2000-NHS using carbodiimide chemistry and characterized using ¹H-NMR. Nanoparticles (LAg-nano or PAg-nano) were prepared with sizes between 200-1000 nm and zeta potentials between (+40 to −60 mV). Both particle types were prepared using the solvent evaporation emulsion method as known in the art. Ag loadings were controlled by admixing biomaterial-Ag conjugates with unmodified PLGA at various ratios. These experiments confirmed that LAg-nano and PAg-nano could be prepared with tunable Ag loadings and well-controlled physicochemical properties through systematic combination of biomaterial-Ag conjugates with unmodified PLGA polymer.

### Antigens

Any protein or peptide antigen can be used with the invention described herein. Preferably, the invention is used with one or more cancer antigens or autoimmune antigens, including MHC I or MHCII antigens.

In certain embodiments, preferred antigens are those which are specifically expressed by a cancer cell type or tumor (including pre-cancers). The invention can moderate the immune response of any cancer for which a specific antigen is known or can be found. Thus, any antigen specific for a tumor or cancer cells is contemplated for use with the invention. In particular, hyperproliferative diseases, such as cancer, are contemplated for use with embodiments of the invention. Therefore, antigens derived from tumor lysates, immunopeptidome (the repertoire of HLA-bound peptides on surface of a cell), multiple antigen peptide (MAP), long peptides, fusion proteins/polypeptides are contemplated for use with certain embodiments of the invention.

In addition, in certain embodiments, autoimmune antigens (i.e., antigens) can be used in this invention in subjects suffering from an autoimmune disease such as multiple sclerosis, celiac disease, inflammatory bowel disease, rheumatoid arthritis, systemic lupus erythematosus, aplastic anemia, and the like.

Antigens suitable for use in the invention include both MHC class I and MHC class II antigens such as proteolipid protein (or peptide), myelin oligodendrocyte glycoprotein (or peptide), gliadins, insulin, p31 peptide, and the like. Preferred antigens include, but are not limited to OVA₃₂₃₋₃₃₉, OVA₂₅₇₋₂₆₄, Ea₅₂₋₆₈, PLP₁₃₉₋₁₅₁, PLP₁₇₈₋₁₉₁, gp100 (20-mer or 9-mer), Prostatic acid phosphatase, HPV, E6/E7, L1, MUC-1, HER2/neu, telomerase, CEA, BCMA and the like for autoimmune, allergy, cancer, or infectious diseases or conditions.

### Lipids

Lipids which can be used in this invention include any lipid containing a fatty moiety such as cholesterol and fatty acids that can insert into the membrane of a cell or a lipid nanoparticle. Such lipids include fatty acids and fatty acid derivatives, triglycerides, and phospholipids. The lipids also can be optionally PEGylated. Suitable fatty acids which form the lipid molecule or part of the lipid molecule can include saturated, monounsaturated, or polyunsaturated fatty chains from about 4 to about 18 carbon atoms. Preferred lipids include 1,2-Distearoyl-sn-glycero-3-phosphorylethanolamine (DSPE), phosphorylethanolamine phospholipid (DSPE)-PEG, Stearic acid-PEG, cholesterol-PEG, distearoylphosphatidylcholine (DSPC)-PEG, and their non-PEGylated counterparts, palmitoyl, lipid A/LPS, and the like, while most preferred lipids are DSPE-PEG. In general, any lipid which are capable of “depoting” into the nanoparticle (a mechanism which mimicks the natural insertion of glycosylphosphatidylinositol (GPI)-anchored proteins into cell membranes) are suitable for use with the embodiments of the invention.

### Nanoparticles

Preferred nanoparticles suitable for use with embodiments according to the invention are composed of poly(lactic-co-glycolic acid), poly(lactic acid), poly(ε-caprolactone), polystyrene, poly(methyl methacrylate), N-(2-hydroxypropyl)-methacrylamide, poly(ethylene glycol), poly(glycolic acid), polyanhydrides, poly(cyano-acrylates), poly(maleic acid), poly(N-vinyl pyrrolidine), chitosan, hyaluronic acid, albumin, heparin, or lipids including but not limited to DSPE-PEG, stearic acid-PEG, cholesterol-PEG, DSPC-PEG, and their non-PEGylated counterparts, or mixtures thereof. Most preferred nanoparticles are composed of poly(lactic-co-glycolic acid) or poly(lactic acid). The nanoparticles can be immunogenic (immune stimulating) or immune tolerizing, depending on the antigen which they contain and their physicochemical properties including nanoparticle composition, surface chemistry, shape, and size.

Nanoparticles suitable for the invention can be from about 10 nm to about 1500 nm in diameter, preferably about 100 nm to about 1200 nm in diameter, more preferably about 200 nm to about 1000 nm in diameter, and most preferably about 400 nm to about 600 nm or about 500 nm.

In certain embodiments of the invention, the zeta potential of the nanoparticles is about −60 mV to about 10 mV, most preferably about +40 mV to about −60 mV.

Preferably, the antigen-lipid conjugates are introduced into the nanoparticles by the following method: emulsification-based methods of nanoparticle formulation using sonication or nanoprecipitation-based methods for nanoparticle formulation or surface conjugation or insertion onto pre-formed nanoparticles. In general, lipo-antigens are dissolved in a suitable organic solvent that is miscible with the polymer solvent at a concentration of about 20 mg/mL, preferably dimethylsulfoxide. The polymer solvent is preferably ethyl acetate or dichloromethane and the polymer is dissolved at a concentration of about 20-200 mg/mL, preferably 50 mg/mL. Emulsion stabilizers useful for this nanoparticle formation are: preferably, poly(vinyl alcohol), poly(ethylene-alt-maleic acid), and others known in the art of nanoparticle formulation. For certain embodiments, the nanoparticles contain from about 0.5 μg of antigen (delivered as lipo-antigen) per mg of nanoparticle to about 300 μg of antigen (delivered as lipo-antigen) per mg of nanoparticle, preferably from about 8 μg of antigen (delivered as lipo-antigen) per mg of nanoparticle to about 150 μg of antigen (delivered as lipo-antigen) per mg of nanoparticle. Suitable amounts of loading include, for example 5 μg Ag/mg, 8 μg Ag/mg, 10 Ag μg/mg, 15 μg Ag/mg, 20 μg Ag/mg, 25 μg Ag/mg, 30 μg Ag/mg, and 50 μg Ag/mg nanoparticle.

### Antigen Presenting Cells

Any cell that can function as an antigen presenting cell is contemplated for use with this invention. In preferred embodiments, however, the antigen presenting cells are macrophages, dendritic cells resting B cells, or activated B cells. The most highly preferred antigen presenting cell for certain embodiments is a resting B cell.

Loading of the APC with antigen using nanoparticles is dose- and time-dependent. Preferably, a solution of nanoparticles containing antigen (LAg-nanos) is contacted with the APC, ex vivo or in vitro. Preferably, the APC are added to a 96-well plate (20,000 cells per well). The nanoparticles are provided to the cells in a solution/suspension of about 0.1 mg/mL to about 250 mg/mL or more in phosphate buffered saline or RPMI 1640 medium containing FBS and penicillin/streptomycin.

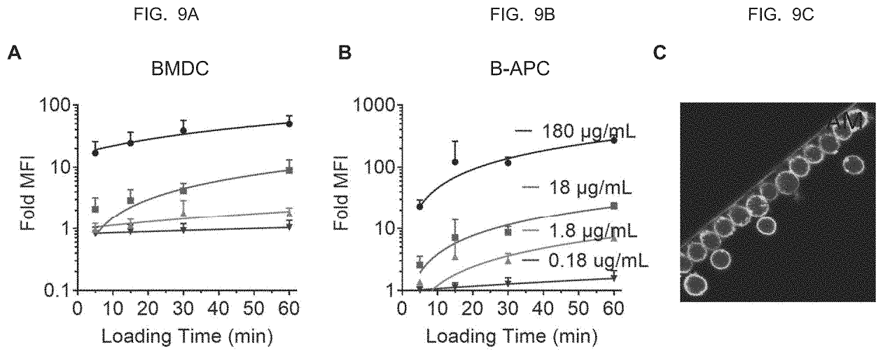

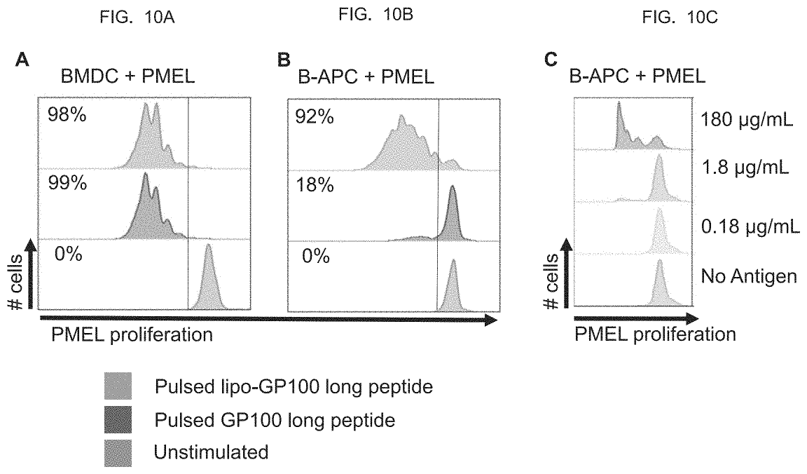

Preferably, for some embodiments, the concentration is about 0.2 mg/mL or more. More preferably, the concentration is about 2 mg/mL to about 200 mg/mL or about 5 mg/mL to about 200 mg/mL, and most preferably about 10 mg/mL to about 200 mg/mL. The nanoparticles are contacted with the APC for about 3 minutes to over 1 hour. In some embodiments, the contact is from about 5 minutes to about 2 hours, or about 10 minutes to about 1 hour, or about 15 minutes to about 1 hour, or about 30 minutes to about 1 hour. Preferably, the contact time is over 30 minutes or over 45 minutes. The most preferred time is 1 hour. Lipo-Ag loading into B cells was dose- and time-dependent, with maximal loading achieved about 1 hour after exposure to Lipo-gp100-FITC, for example. Therefore, the practitioner can determine a convenient time and concentration. See FIG. 9 and FIG. 10.

### Subjects

Any mammalian subject in need is contemplated for use with the invention. Preferably, the subject is human, but other mammals, including laboratory animals (e.g., mice, rats, rabbits, and the like), farm animals and livestock (e.g., cattle, horses, sheep, goats, and the like), companion animals (e.g., dogs, cats, and the like) also are contemplated for use with the invention. The subject, in some embodiments, suffers from a hyperproliferative disorder such as cancer in any organ or tissue, or suffers from an autoimmune condition. In general, any condition which would be ameliorated, improved, halted in its progression, or retarded by modulation of the immune response to a specific antigen or antigens is suitable for use. Further, any subject that would benefit from modulation of their immune response to a particular antigen or antigens is suitable for administration of the inventive nanoparticles and APCs. Thus, any suitable antigen or suitable APC can be used with the invention, depending on the need.

### Disease Conditions

The invention can be used to produce nanoparticles and antigen presenting cells, especially B cells, that are specific for any protein or peptide antigen. Particularly preferred antigens are those which produce a specific immune response to cancers (i.e., cancer antigens), pathogens, infectious diseases, to tolerize to autoimmune diseases (i.e., autoimmune antigens), or tolerize to allergies (i.e. allergens). Specific cancers that are contemplated for the invention include, but are not limited to breast, prostate, melanoma, lung, pancreatic, lymphoma, glioblastoma, head and neck cancer, leukemia, and myeloma. Thus, cancer antigens that are useful for embodiments of the invention include, but are not limited to gp100 or preferably, tumor cell lysates, and the like.

The invention also can be useful in vaccine development, for example for infectious diseases such as sepsis (viral, bacterial, fungal, or parasitic), or hepatitis B. Thus, additional antigens that can be used with the invention include antigens specific to disease organisms or diseases such as cell lysates, and suitable antigens include, but are not limited to HBsAg, and the like.

### Routes of Administration and Doses

The compositions and pharmaceuticals according to this invention can be administered to a subject in need by any route considered suitable and convenient by the medical practitioner or laboratory scientist. Preferably, nanoparticles or antigen presenting cells are administered to a subject intravenously. Additional routes of administration which can be useful include, intraarterial injection, local injection to the area of disease (such as into a tumor or affected tissue), intracerebral, intrathecal, intraperitoneal, intravenous, intraorbital, intranodal, intrahepatic, intrasplenic, and the like.

Doses to be administered to a subject can be determined by the person of skill depending on the size and condition of the subject, the disease or condition to be treated, and the like. Generally, however, it is anticipated that doses of nanoparticles will be about 100 mg to about 1 g and preferably about 250 mg to about 500 mg. Doses to be administered to a subject can be determined by the person of skill depending on the size and condition of the subject, the disease or condition to be treated, and the like. Generally, however, it is anticipated that doses of nanoparticles will be about 100 mg to about 1 g and preferably about 250 mg to about 500 mg.

### 5. EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: Materials and Methods

### Materials

Poly(lactide-co-glycolide) (50:50) (PLGA) with a single carboxylic acid end-group and an inherent viscosity of 0.17 dL/g in hexafluoro-2-propanol was purchased from Lactel Absorbable Polymers™. DSPE-PEG2000-NHS (Lipid) was purchased from Nanocs™. Anionic polyelectrolyte poly(ethylene-alt-maleic anhydride) (PEMA) was purchased from Polyscience™, Inc. (Warrington, Pa.). Peptide Antigens, including amine-terminated OVA₂₅₇₋₂₆₄ (SIINFEKL; SEQ ID NO:1) (termed OTIAg), OVA₃₂₃₋₃₃₉ (ISQAVHAAHAEINEAGR; SEQ ID NO:2) (termed OTIIAg) , amine-terminated OVA peptide Ags (OTAgs), and GP100 (CAVGALEGPRNQDWLGVPRQL; SEQ ID NO:3) were purchased from GenScript Biotech™. Fluorescein-labeled SIINFEKL (SEQ ID NO:1) was purchased from Anaspec™. All other reagents were purchased from Sigma Aldrich™ except as noted otherwise.

### PLGA- and Lipo-Ag Conjugation Methods

PLGA (37.8 mg, 0.009 mmol, 4200 g/mol) was dissolved in 2 mL of dimethylsulfoxide (DMSO) in a 20 mL scintillation vial equipped with a stir bar. N-(3-Dimethylaminopropyl)-N′-ethylcarbodiimide hydrochloride (EDC) (9.0 mg, 0.047 mmol, 5 times molar excess to PLGA) was dissolved in 0.5 mL of DMSO and added dropwise to the PLGA solution. N-Hydroxysuccinimide (NHS) (5.5 mg, 0.047 mmol, 5 times molar excess to PLGA) was dissolved in 0.5 mL DMSO and added dropwise to the solution. The reaction was allowed to stir for 15 minutes at room temperature. Antigenic peptides (1.1 times molar excess to PLGA) were dissolved in a solution of 1 mL dimethyl sulfoxide (DMSO) and stirred at 400 RPM.

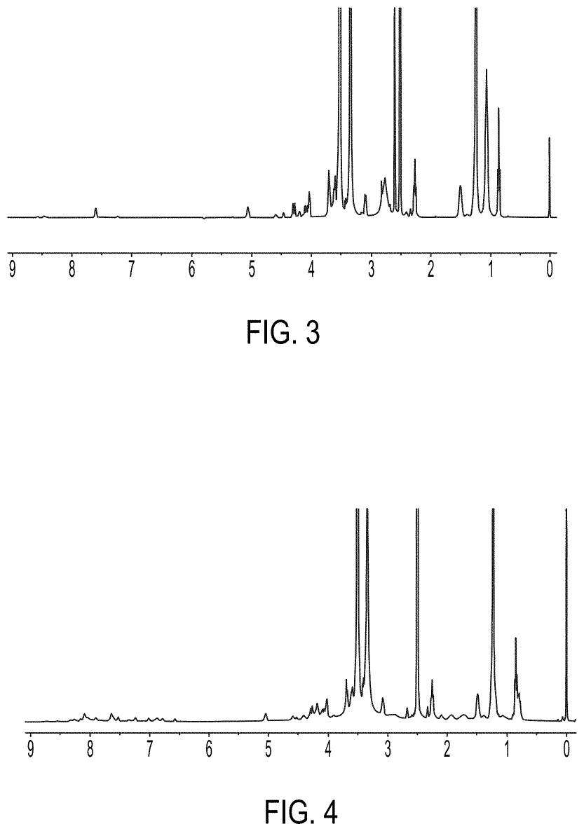

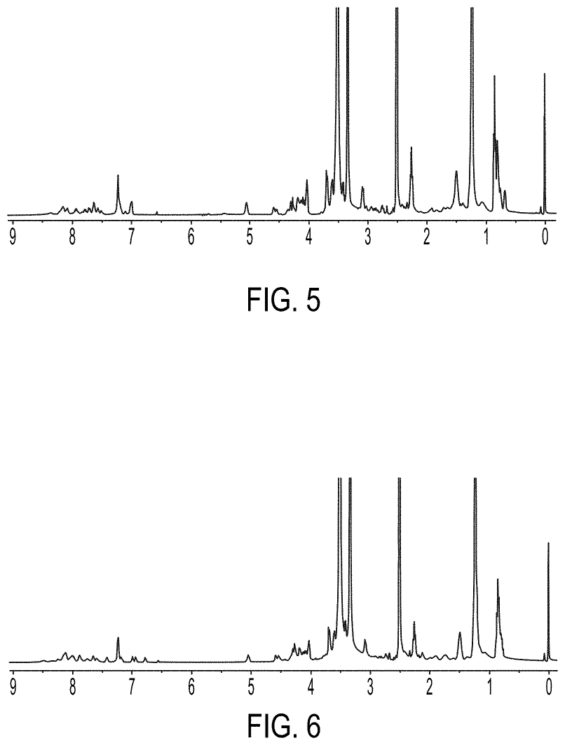

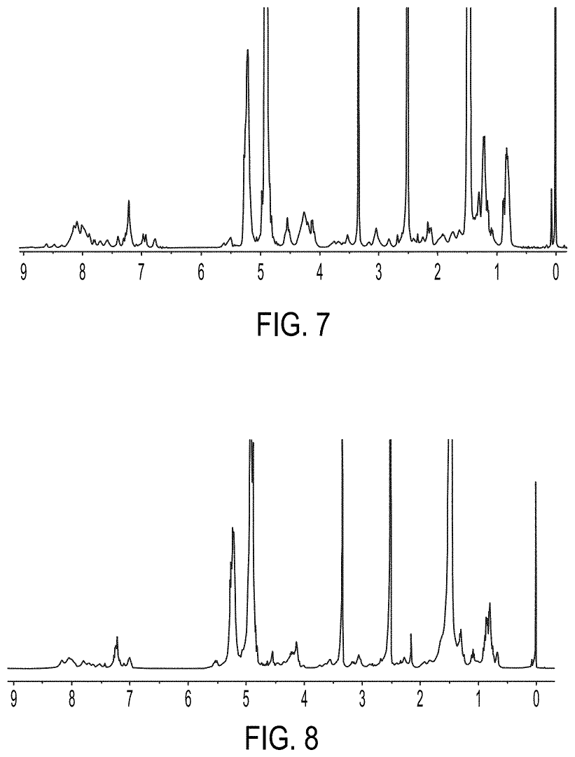

Triethylamine (5 times molar excess to peptide) was added to the peptide solution and the mixture was added dropwise to the stirring PLGA solution. The reaction was allowed to proceed overnight at room temperature. The resulting polymer was isolated and purified by dialysis using a 3,500 molecular weight cut-off membrane against four liters of distilled water over two days. The distilled water was replaced a total of six times. The dialyzed polymer was collected and washed with MilliQ™ water three times by centrifugation at 7000×g before resuspension in 20 mL of water and lyophilization for two days. Coupling efficiency peptide to PLGA was determined by ¹H-NMR analysis in DMSO-d6. See FIGS. 3-8.

DSPE-PEG2000-NHS (Lipo) (20 mg, 0.0071 mmol, 2800 g/mol) was dissolved in 2 mL of DMSO in a 20 mL scintillation vial with a stir bar and placed on a stir plate at 500 RPM. Peptides (1.1 times molar excess to DSPE-PEG2000-NHS were dissolved in 0.5 mL DMSO in a 1.5 mL microcentrifuge tube along with triethylamine (5× times molar excess to peptide). Under stirring, the peptide/TEA mixture was added to the DSPE-PEG2000-NHS solution dropwise to form Lipo-Ag. The reaction was allowed to proceed overnight at room temperature. The resulting conjugation was recovered by dialysis using a 3.5 kD weight cut off membrane against 4 L of distilled water. See FIGS. 3-8.

The membrane was placed in a 250 mL beaker with MilliQTM water (fully submerged) and soaked for 5 minutes. One end of the membrane (at least 1 inch from the end) was closed with a clip and the other end rubbed open. The reaction was slowly pipetted into the membrane, and the end closed at least 1 inch from the top. The length of empty membrane should be at least 2 times the height of the sample in the dialysis bag to avoid bursting. The water was changed a total of 6 times over 2 days (6 exchanges). To collect the product, the solution was transferred from the dialysis bag to a 20 mL scintillation vial (careful not to fill the vial more than half full to avoid breaking upon expansion) and frozen at −80° C. The cap was left partially open (also to allow for water expansion when freezing). The container was covered with foil with holes and lyophilized for 2 days.

### Mice

C57BL/6J (6-8 weeks old), OTI (C57BL/6-Tg(TcraTcrb)1100Mjba (6-8 weeks old), OT-II (B6.Cg-Tg(TcraTcrb)425 Cbn/J) (6-8 weeks old), and PMEL (B6.Cg-Thy1^{a}/CyTg(TcraTcrb)8Rest/J) (6-8 weeks old) mice were used. Mice were purchased from Jackson Laboratory™ (Bar Harbor, Me.) or bred according to approved animal protocols. All mice were housed under specific pathogen-free conditions.

### Cell Culture

Mouse bone marrow-derived dendritic cells (BMDC) were generated from the bone marrow of C57BL/6J mice using the Lutz protocol known in the art. Media consisted of RPMI containing L-glutamine (Life Technologies™, Carlsbad, Calif.), supplemented with penicillin (100 units/mL), streptomycin (100 mg/mL), 10% heat-inactivated fetal bovine serum (FBS) (Invitrogen™ Corporation, Carlsbad, Calif.) and 50 mM β-mercaptoethanol (Sigma Aldrich™). GM-CSF (Peprotech™, Rocky Hill, N.J.) was added at 20 ng/mL, and media was added on days 3, 6, and 8. T and B cell media was similar but without GM-CSF or β-mercaptoethanol and supplemented with 1 mM sodium pyruvate, and 0.1 mM non-essential amino acids (Life Technologies™, Carlsbad, Calif.).

### Cell Isolation and in vitro Co-Culture Assays

B cells were isolated using magnetic activated cell sorting (MACS) using a mouse B cell isolation kit following procedures provided by the manufacturer (Miltenyi Biotec™, Waltham, Mass.). Naive T cells were isolated from the spleens of OTI, OTII, and PMEL mice using a mouse T cell isolation kit (Miltenyi Biotec™). Isolated T cells were labeled with 5 μM of carboxyfluorescein succinimidyl ester (CFSE, ThermoFisher™) and cultured in T media described above.

T cell proliferation studies assays were carried out as described. The assay was carried out in T cell media. BMDCs (2×10⁴/well) or B cells (2×10⁴/well) were seeded into 96-well round-bottom cell culture plates and incubated with LAg-nano or PAg-nano at various particle concentrations and Ag loadings for 3 hours. Following incubation, all wells were washed to remove excess particles that had not been internalized by cells. Cells were co-cultured with 2×10⁴/well of each OTI (CD8) and OTII (CD4) T cells). Groups receiving antigen in lieu of LAg-nano or PAg-nano received soluble OVA₃₂₃₋₃₃₉ or SIINFEKL (SEQ ID NO:1; 100 ng/mL) at this time. After 3 days of co-culture, the T cells were collected, stained for viability, CD4, and CD8 and analyzed using flow cytometry. The extent of T cell proliferation was analyzed by CFSE dilution using flow cytometry. Ag processing kinetics in B cells was determined by loading with long GP100, KVPRNQDWL (SEQ ID NO:4; short GP100), or lipid-long GP100, CAVGALEGPRNQDWLGVPRQL (lipid-GP100; SEQ ID NO:3). B cells were washed, then rested for 0, 3, or 16 hours before fixation. Fixed B cells were co-cultured with PMEL T cells for T-cell proliferation analysis.

### Example 2. Nanoparticle Preparation and Loading

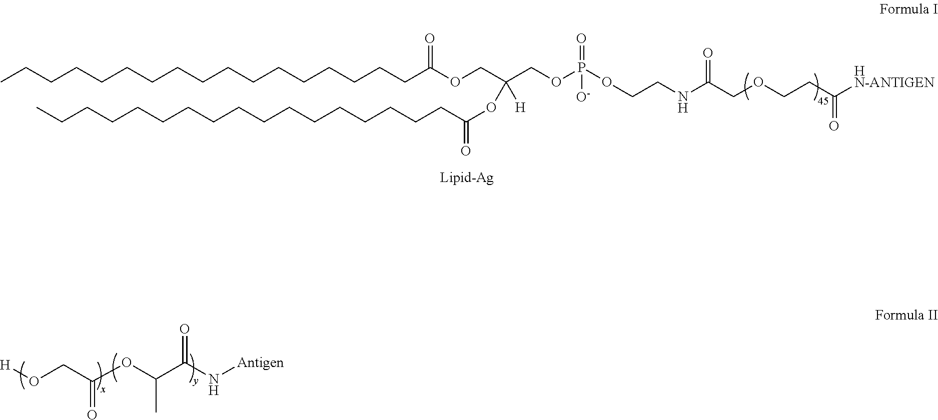

Nanoparticles (PAg-nano and LAg-nanos) were prepared following the emulsion solvent evaporation method. To produce nanoparticles using this method, biomaterial-Ag conjugates (poly-Ag or Lipo-Ag) were combined with unmodified PLGA at various ratios to give calculated Ag loadings in particles. Cryoprotectants (4% (w/v) sucrose and 3% (w/v) mannitol) were then added to the particles before lyophilization. See above Formula I for the chemical structure of DSPE-PEG2000-Ag conjugates (Lipid-Ag). See above Formula II for the chemical structure of PLGA-Ag conjugates (PLGA-Ag), where x=16 and y=16.

The size and zeta potential of the PAg-nano and LAg-nanos were determined by dynamic light scattering (DLS) by mixing 10 μL of a 25 mg/mL particle solution into 990 μL of MilliQ water using a Malvern Zetasizer ZSP (Westborough, Mass.).

TABLE 1
Antigens

| MHC class I | MHC class II |
|---|---|
| SIINFEKL; | Ea52-68; |
| SEQ ID NO: 1 | SEQ ID NO: 5 |
| Gp100 | OVA323-339; |
| | SEQ ID NO: 2 |

OVA protein

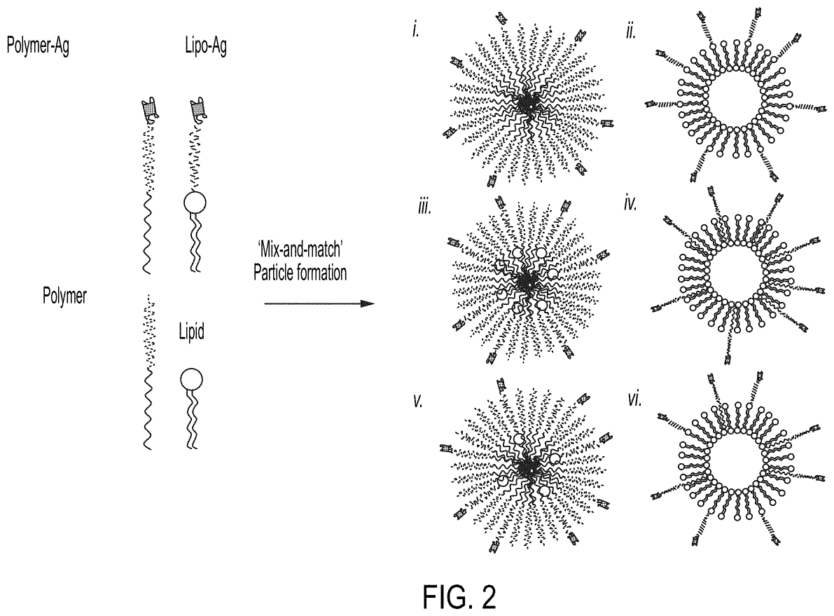

A major challenge in immunotherapy has been enabling the delivery of therapeutic cargo to specifically and effectively mitigate aberrant immune activation in diseases. Lipid-tailed Ags (Lipo-Ags) insert with high efficiency into immune cell plasma membranes. The insertion of Lipo-Ags into membranes is similar to the natural insertion of GPI-anchored proteins in that they can insert into splenocytes and resting lymphocytes (B cells, CD4⁺ T cells, CD8⁺ T cells, and other traditional APCs). See FIG. 1 and FIG. 2.

FIG. 1 shows the LAg-nano as a novel immune-modulating platform for the induction of Ag-specific immune responses in CD4⁺ and CD8⁺ T cells in schematic form, including the LAg-nano formulation and representation of 2 types of cargo delivery mechanisms. FIG. 2 shows a modular approach for formulating various LAg- and PAg-nano nanoparticles described herein. Various combinations of Polymer-Ag, Lipid-Ag, polymer, or lipid are combined in a modular, ‘mix-and-match’ approach to formulate a variety of nanoparticles for the delivery of antigenic cargoes.

### Example 3. Antigen Loading

1,2-distearoyl-sn-glycero-3-phosphoethanolamine (DSPE)-poly(ethylene glycol) (PEG) 2000 was coupled to the N-terminal cysteine of a melanoma Ag long peptide, a class I restricted epitope from gp100. Mouse bone marrow-derived dendritic cells (BMDCs) matured in GM-CSF for 8-10 days and splenic B cells were loaded with fluorescein-labeled lipo-GP100. Using a fluorescently (FAM)-labeled long peptide, the efficiency and dynamics of loading APCs with this Ag under different concentrations, volumes, cell types (dendritic cells and B cells), and time was examined See FIG. 9. FIG. 9A and FIG. 9B show the relative density of lipo-GP100 loading on CD11c⁺ BMDCs and B220⁺ B cells is dependent on loading concentration and time, as analyzed by fold median-fluorescent intensity (MFI) with flow cytometry. Data showed mean ±s.d. n=3-4 independent samples. FIG. 9C shows data on Lipo-GP100 loading on B cells, also analyzed cells by confocal microscopy.

Lipo-Ag loading into B cells was dose- and time-dependent, with maximal loading achieved about 1 hour after exposure to Lipo-gp100-FITC. These results supported that Lipo-Ags could be loaded at significant quantities into B cell membranes.

### Example 4. APC Internalization and MHCI-Restricted Antigen Presentation

Lipo-Ag conjugate enables APC internalization and promotes MHC I-restricted antigen presentation to cognate CD8⁺ T cells. FIG. 10A and FIG. 10B present data for Lipo-GP100 (20-mer) loaded onto BMDCs and B cells and functionally presented by APCs to GP100-specific CD8⁺ T cells (PMELs) similar to minimal GP100 (9-mer) presentation, as determined by CFSE dye dilution after 3-day co-culture. Percentages represent percent of divided PMELs. FIG. 10C shows that Lipo-GP100 internalization and presentation by B cells is concentration-dependent.

Delivery of lipid-GP100 induced BMDCs and B cells to process and present MHCI-restricted epitope to PMEL CD8+ T cells compared to that of unmodified long GP100. Processing and presentation of MHCI-restricted lipid-GP100 was prolonged up to 16 hours compared to short GP100 at 16 hours. See FIG. 10.

### Example 5. APC Internalization and MHCII-Restricted Presentation

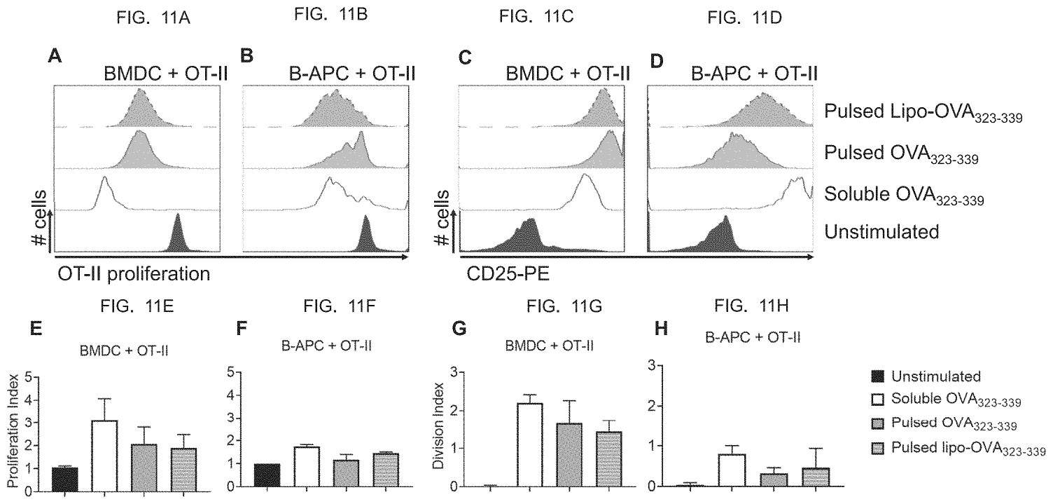

Lipo-Ag enables APC internalization and promotes MHC II-restricted presentation to cognate CD4⁺ T cells. BMDCs and B cells loaded with unmodified ovalbumin (OVA) MHC II-restricted antigen (OVA₃₂₃₋₃₃₉) or lipo-OVA₃₂₃₋₃₃₉ were co-cultured with OVA-specific CD4⁺ T (OT-II) cells for 3 days. CD25+ expression was detected and the proliferation and division indices were calculated. Representative OT-II cell activation as shown by CFSE dye dilution (FIG. 11A and FIG. 11B) and CD25 expression (FIG. 11C and FIG. 11D) using flow cytometry. FIGS. 11E through 11H show quantitation of the proliferation and division indices of OT-II T cells. Data showed are mean ±s.d. (n=2 independent samples).

Thus, Lipo-OVA₃₂₃₋₃₃₉ enables APC internalization and promotes MHC II-restricted presentation to cognate OTII CD4⁺ T cells.

### Example 6. Antigen Presentation in Selected Cells

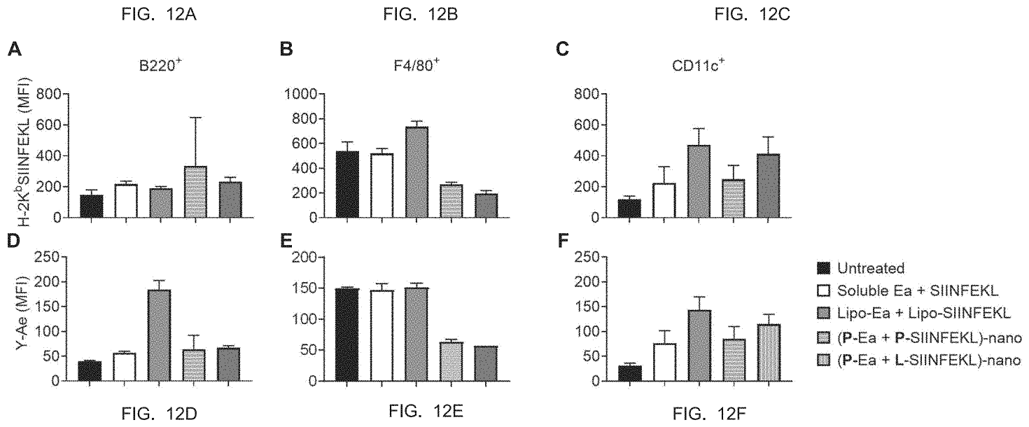

Subsequently, the antigen presentation capabilities of LAg-nano and PAg-nanos were assessed to compare to Lipo-Ag conjugates for B cells, macrophages, and dendritic cells using flow cytometry. See FIG. 12. For the data in this figure, mouse bone marrow-derived cells were matured in M-CSF or GM-CSF for 8-10 days for respective F4/80+ or CD11c+ cell lineage generation.

Lipo-Ag conjugates and LAg- +PAg-nano were loaded onto APCs and presented on surface MHCs. LAg- +PAg-nano loaded (SIINFEKL (SEQ ID NO:1; MHCI) or Ea52-68 (ASFEAQGALANIAVDKA; SEQ ID NO:5; MHCII)) onto APCs and are presented on surface MHCs. Presentation of SIINFEKL (SEQ ID NO:1) on MHC I and Ea on MHC II was determined by MFI with flow cytometry. B220+ (FIG. 12A and FIG. 12D), F4/80+ (FIG. 12B and FIG. E), and CD11c+ (FIG. 12C and FIG. 12F) APCs were loaded with SIINFEKL (SEQ ID NO:1) and Ea peptides as lipo-Ag conjugates or L/P-Ag PLGA NPs, (P-Ea+P- SIINFEKL (SEQ ID NO:1))-nano or (P-Ea+L-SIINFEKL)-nano. Presentation of SIINFEKL (SEQ ID NO:1) on MHC I and Ea on MHC II was respectively determined by MFI with flow cytometry. P denotes PLGA polymer (PAg-nano), L denotes Lipid (LAg-nano). Data showed mean ±s.d. n=3 replicates.

Lipid conjugated Ags and LAg-nanos were most effective at inducing high levels of MHCI or MHCII antigen presentation.

### Example 7. Presentation by Dendritic Cells

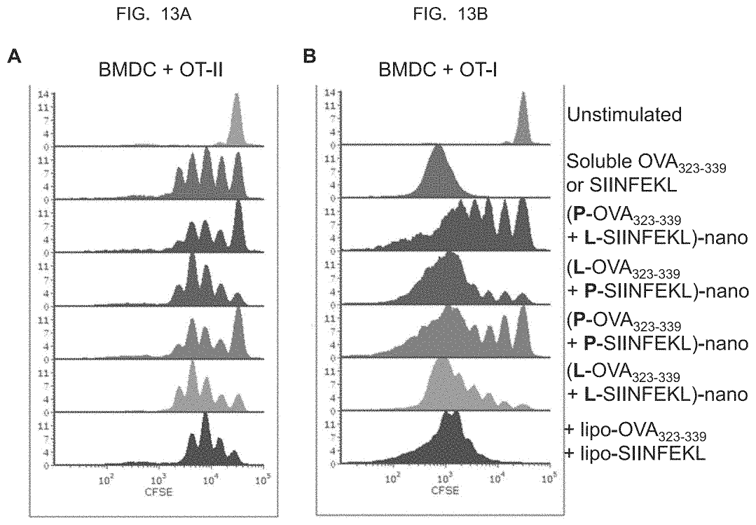

Lipo-Ag conjugates and P/L-Ag-nano enable MHC I- and MHC II-restricted presentation by BMDCs for priming cognate CD4⁺ (OT-II) and CD8⁺ (OT-I) T cells. BMDCs were loaded with OVA-derived Ags as lipo-Ag conjugates, or P/L-Ag blends in NPs, (P-OVA₃₂₃₋₃₃₉+L-SIINFEKL (SEQ ID DNO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL(SEQ ID NO:1))-nano, and co-cultured with OT-II (FIG. 13A) and OT-I cells FIG. 13B) for 3 days. Representative histograms show T-cell proliferation as determined by CFSE dye dilution using flow cytometry. P denotes PLGA polymer (PAg-nano), L denotes Lipid (LAg-nano).

DCs loaded with Lipo-Ag, LAg-nano, and PAg-nano conjugates presented both MHCI (SIINFEKL; SEQ ID NO:1) and MHCII (SEQ ID NO:2; OVA₃₂₃₋₃₃₉)-restricted epitopes and proliferated Ag-specific T-cells isolated from OTI and OTII spleens (see FIG. 13).

### Example 8. Presentation by B Cells

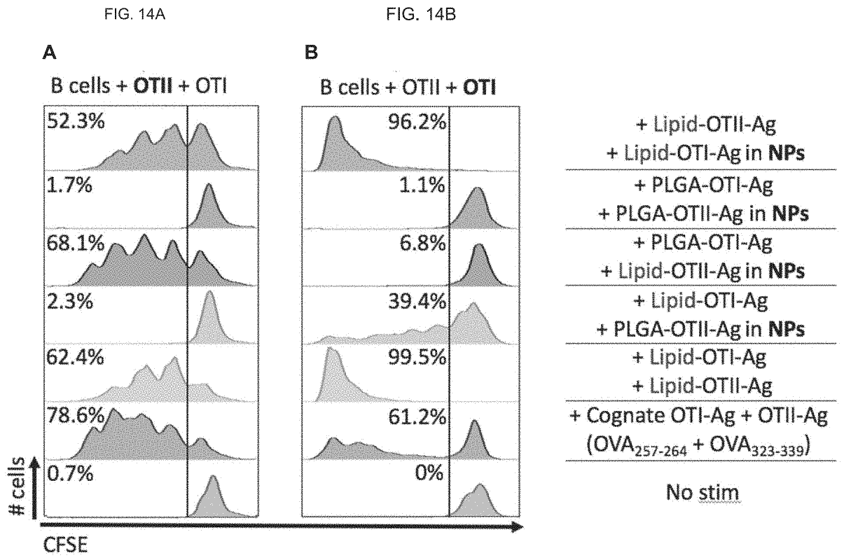

Lipo-Ag conjugates and P/L-Ag-nano enable MHC I- and MHC II-restricted presentation by B cells for priming cognate CD4⁺ (OT-II) and CD8⁺ (OT-I) T cells. B cells were loaded with OVA-derived Ags as lipo-Ag conjugates, or P/L-Ag blends in NPs, (P-OVA₃₂₃₋₃₃₉+L-SIINFEKL (SEQ ID NO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL(SEQ ID NO:1))-nano, and co-cultured with OT-II (FIG. 14A) and OT-I (FIG. 14B) cells for 3 days. Representative histograms show T-cell proliferation as determined by CFSE dye dilution using flow cytometry. P denotes PLGA polymer (PAg-nano), L denotes Lipid (LAg-nano).

While DCs efficiently internalized both LAg-nanos and PAg-nanos, B cells preferentially processed Ag delivered by LAg-nanos compared to PAg-nanos and induced Ag-specific T-cell priming (see FIG. 14).

### Example 9. Effects of Lipid Conjugation on T Cell Proliferation

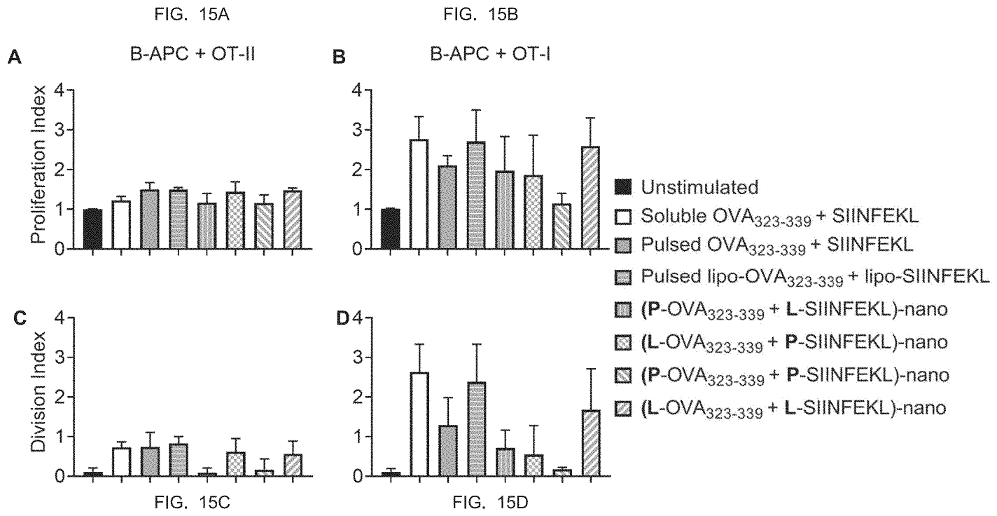

Lipid-based biomaterials enable MHC I- and MHC II-restricted presentation by B cells for priming cognate CD4⁺ (OT-II) and CD8⁺ (OT-I) T cells. B cells were loaded with OVA-derived Ags as lipo-Ag conjugates, or P/L-Ag blends in NPs, (P-OVA₃₂₃₋₃₃₉+L-SIINFEKL (SEQ ID NO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL (SEQ ID NO:1))-nano, and co-cultured with OT-II (FIG. 15A and FIG. 15C) and OT-I cells (FIG. 15B and FIG. 15D) for 3 days. T-cell proliferation as determined by CFSE dye dilution using flow cytometry, and quantitation of proliferation and division indices was determined. P denotes PLGA polymer (PAg-nano), L denotes Lipid (LAg-nano). Data showed mean ±s.d (n=2-4 independent samples).

Lipid conjugation to Ags did not affect their ability to induce CD4⁺ or CD8⁺ T cell proliferation compared to controls. The division and proliferation indices were calculated for B cells and corresponding MHCI and MHCII antigens (see FIG. 15).

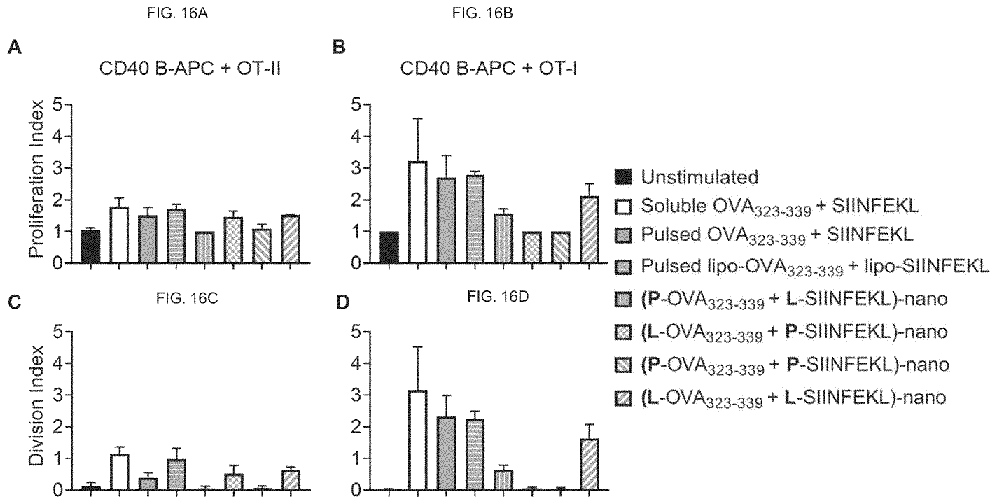

A similar experiment to assess antigen presentation was carried out for active CD40 B cells (see FIG. 16). Splenic B cells were activated with CD40 mAb and R848 agonist for 2 days. Activated B-APC (CD40 B-APCs) were loaded with OVA-derived Ags as lipo-Ag conjugates, or P/L-Ag blends in NPs, (P-OVA₃₂₃₋₃₃₉+L-SIINFEKL (SEQ ID NO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL (SEQ ID NO:1))-nano, and co-cultured with OT-II (FIG. 16A and FIG. 16C) and OT-II (FIG. 16B and FIG. 16D) cells for 3 days. T-cell proliferation as determined by CFSE dye dilution using flow cytometry, and quantitation of proliferation and division indices was performed. P denotes PLGA polymer (PAg-nano), L denotes Lipid (LAg-nano). Data shown include mean ±s.d (n'2 independent samples).

Lipid-based biomaterials enable MHC I- and MHC II-restricted presentation by CD40 B cells for priming cognate OT-II and OT-I T cells.

### Example 10. MHC I- and MHC II-Restricted Presentation by B and CD40 B Cells

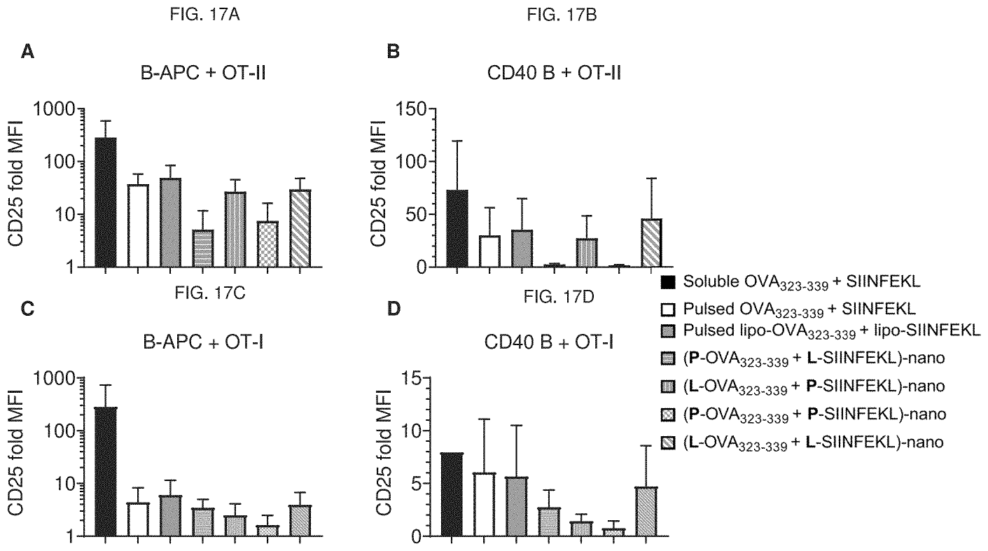

Lipid-based biomaterials enable MHC I- and MHC II-restricted presentation by B and CD40 B cells for priming cognate OT-II and OT-I T cells. B cells and CD40 B cells were loaded with OVA-derived Ags as lipo-Ag conjugates, or P/L-Ag blends in PLGA NPs, (P-OVA₃₂₃₋₃₃₉+L-SIINFEKL (SEQ ID NO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL (SEQ ID NO:1))-nano, and co-cultured with OT-I (FIG. 17A and FIG. 17C) and OT-II (FIG. 17B and FIG. 17D) cells for 3 days. OT-I and OT-II cell activation as determined by CD25 expression using flow cytometry, and quantitation of fold MFI (normalized to unstimulated) was determined. P denotes PLGA polymer (PAg-nano), L denotes Lipid (LAg-nano). Data showed is the mean ±s.d (n=1-5 independent samples).

### Example 11. Cytokine Secretion Profiles

Biomaterial conjugates drive differential cytokine secretion profiles. BMDCs, B cells, and CD40 B cells were loaded with OVA-derived Ags as lipo-Ag conjugates, or P/L-Ag blends in PLGA NPs, (P-OVA₃₂₃₋₃₃₉+L-SIINFEKL (SEQ ID NO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL (SEQ ID NO:1))-nano, and co-cultured with OTI and OTII cells for 3 days.

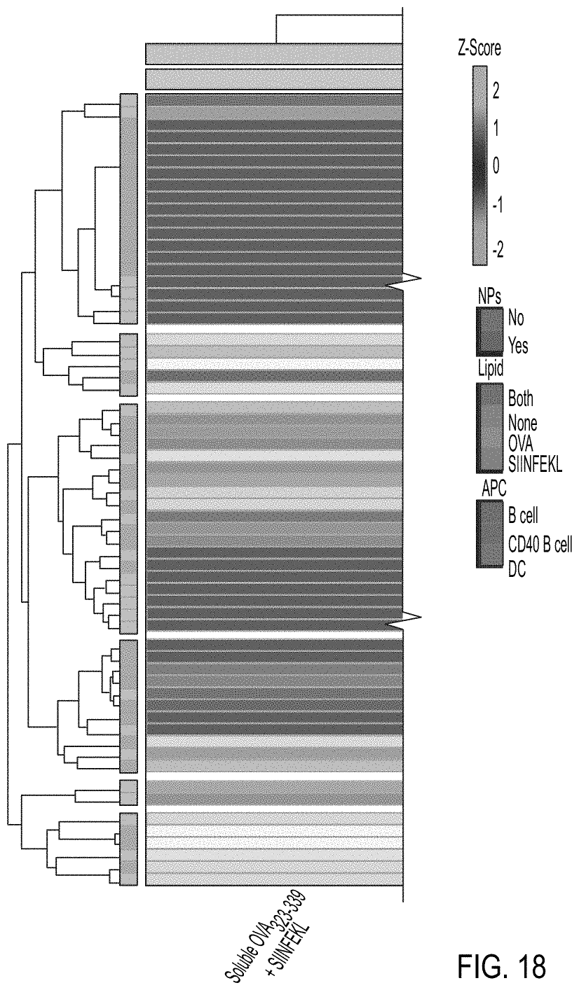

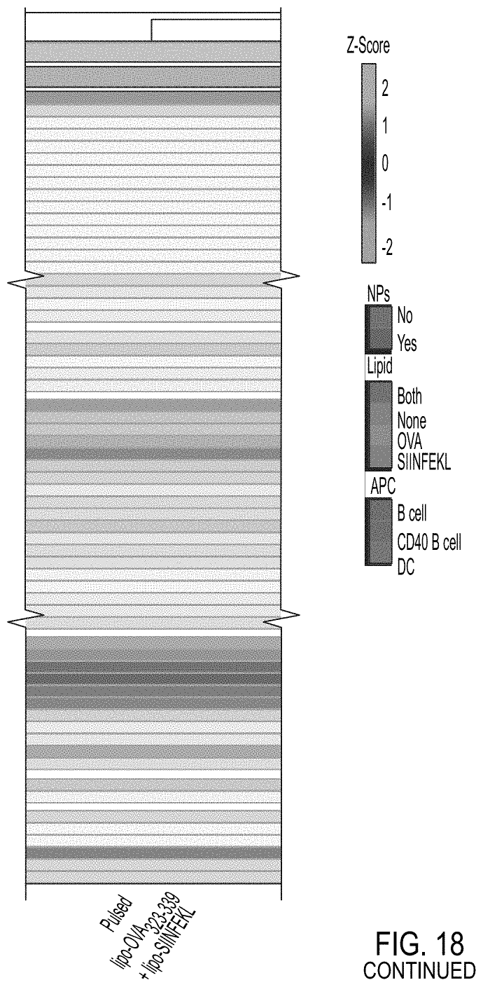

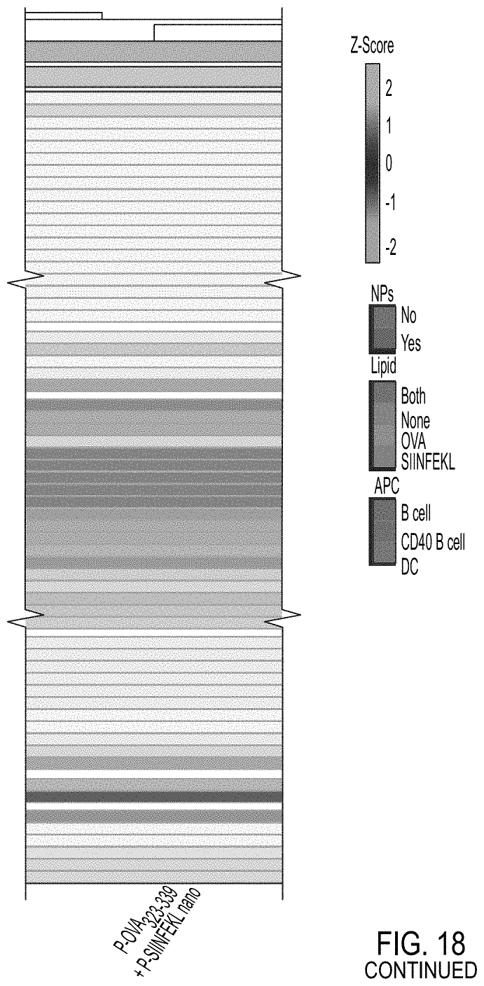

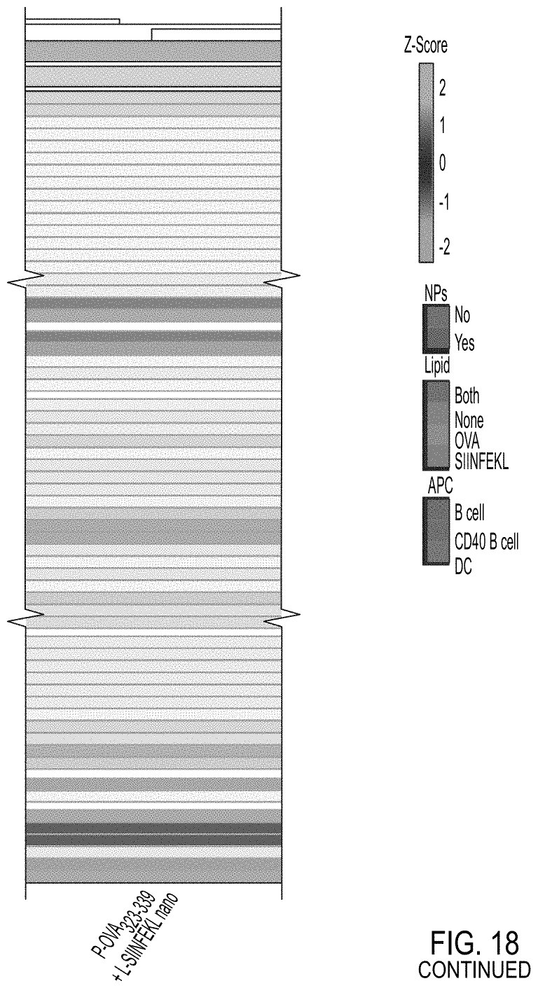

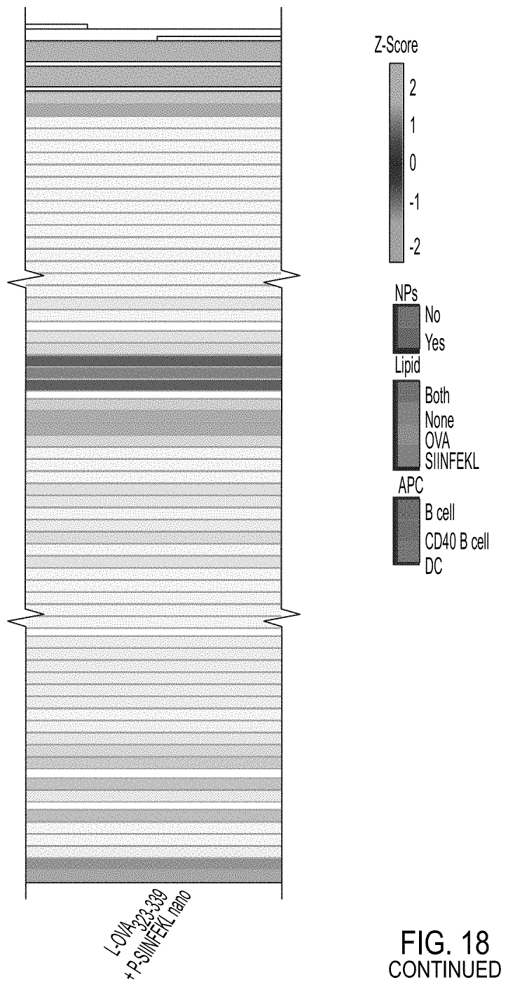

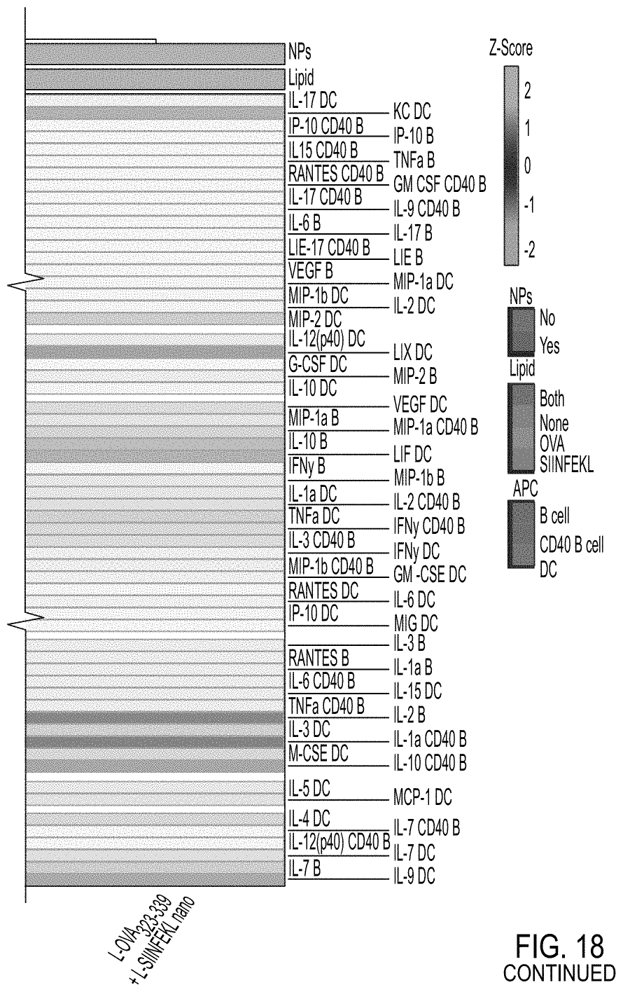

Differential cytokine secretion profiles were measurable for BMDCs, B cells, and CD40 B cells coculture with OTI and OTII T cells. Thirty-two cytokines were measured for each biomaterial-based Ag treatment using Luminex and Z-scores are presented in FIG. 18. The heatmap represents normalized concentrations (z-scores) of 32 cytokines for each biomaterial-based Ag treatment as measured by Luminex P denotes PLGA polymer (PAg-nano), L denotes Lipid (LAg-nano)(n=2 replicates).

### Example 12. Inflammatory Cytokine Levels

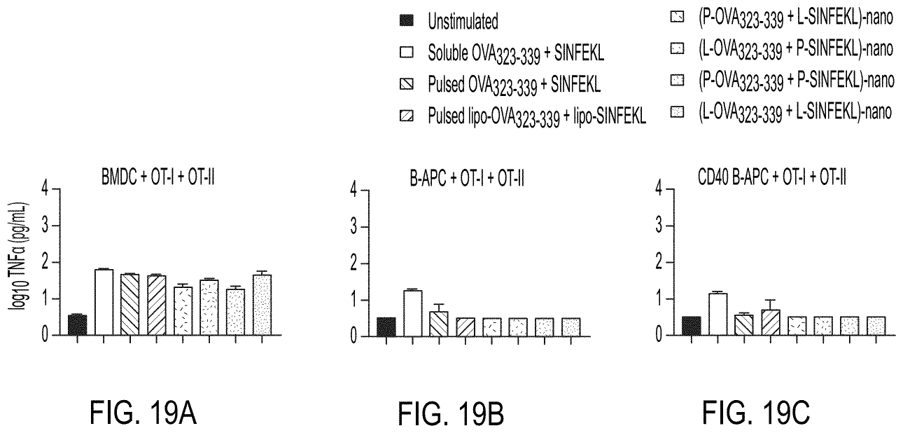

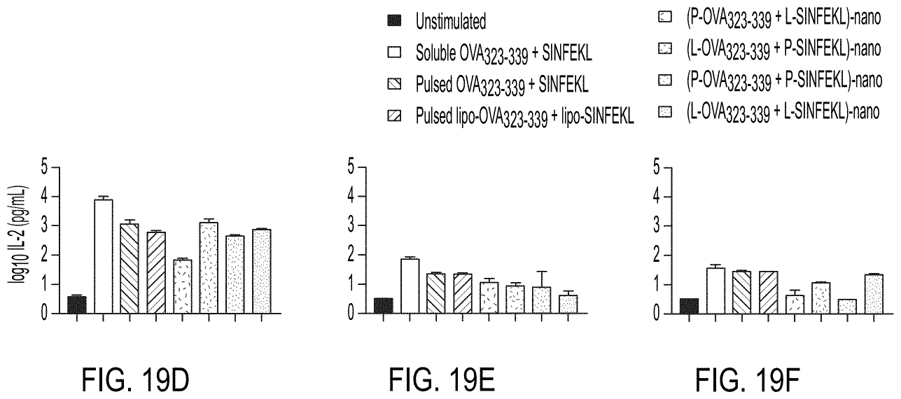

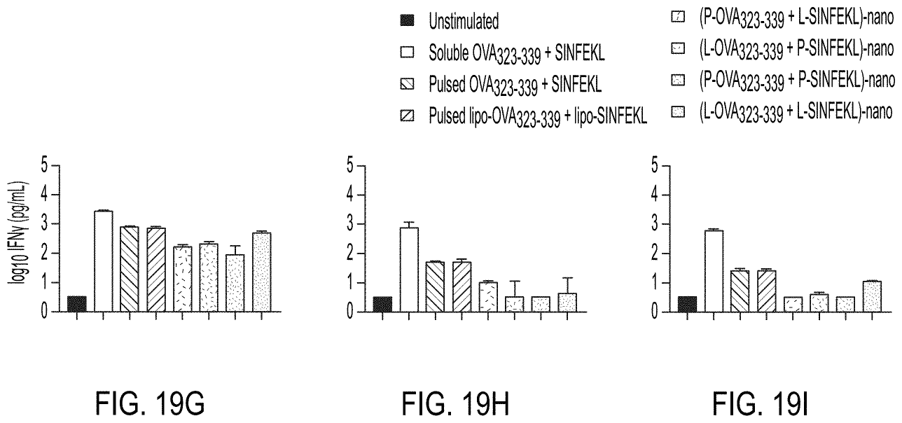

Inflammatory cytokine levels were determined for TNFα, IL-2, and IFNγ for each biomaterial-based treatment. BMDCs, B-APCs, and CD40 B-APCs were loaded with OVA-derived Ags as lipo-Ag conjugates, or P/L-Ag blends in PLGA NPs, (P-OVA₃₂₃₋₃₃₉+L-SIINFEKL (SEQ ID NO:1), L-OVA₃₂₃₋₃₃₉+P-SIINFEKL (SEQ ID NO:1), P-OVA₃₂₃₋₃₃₉+P-SIINFEKL (SEQ ID NO:1), or L-OVA₃₂₃₋₃₃₉+L-SIINFEKL (SEQ ID NO:1))-nano, and co-cultured with OT-II and OT-I cells for 3 days. TNFα, IL-2, and IFNγ cytokine levels for each biomaterial-based treatment were measured by Luminex as indicated in FIGS. 19A through FIG. 19I. P denotes PLGA polymer (PAg-nano), L denotes Lipid (LAg-nano). Data shown are the mean ±s.d. (n=2 replicates).

Lipo-Ag conjugates and P/L-Ag-nano differentially enable MHC I- and MHC II-restricted presentation by APCs and induce pro-inflammatory cytokine secretion.

### REFERENCES

All references listed below and throughout the specification are hereby incorporated by reference in their entirety.

### 1. Casey et al., Cargo-Less Nanoparticles Program Innate Immune Cell Responses to Toll-Like Receptor Activation, Biomaterials, 218 (2019) 119333.

2. Gammon and Jewell, Engineering Immune Tolerance with Biomaterials, Adv Healthc Mater, (2019) e1801419.
3. Greenfield and Hauser, B-Cell Therapy for Multiple Sclerosis: Entering an Era, Ann. Neurol., 83 (2018) 13-26.
4. Hong et al., B Cells Are the Dominant Antigen-Presenting Cells that Activate Naive CD4(+) T Cells upon Immunization with a Virus-Derived Nanoparticle Antigen, Immunity, 49 (2018) 695-708 e694.
5. Hlavaty et al., Tolerance induction using nanoparticles bearing HY peptides in bone marrow transplantation, Biomaterials, 76 (2016) 1-10.

### 6. Hunter et al., A Biodegradable Nanoparticle Platform for the Induction of Antigen-Specific Immune Tolerance for Treatment of Autoimmune Disease, ACS Nano, 8 (2014) 2148-2160.

7. Kuo et al., Peptide-Conjugated Nanoparticles Reduce Positive Co-Stimulatory Expression and T Cell Activity to Induce Tolerance, Mol. Ther., (2017) 1675-1685.
8. Lehmann-Horn et al., Deciphering the Role of B Cells in Multiple Sclerosis-Towards Specific Targeting of Pathogenic Function, Int. J. Mol. Sci., 18 (2017). Liu et al., Structure-Based Programming of Lymph-Node Targeting in Molecular Vaccines, Nature, 507 (2014) 519-522.
9. Lutz et al., An advanced culture method for generating large quantities of highly pure dendritic cells from mouse bone marrow, J. Immunol. Methods, 223 (1999) 77-92.
10. McCarthy et al., An Antigen-Encapsulating Nanoparticle Platform for Th1/17 Immune Tolerance Therapy, Nanomed. Nanotechnol., 13 (2017) 191-200.
11. Miller et al., Antigen-Specific Tolerance Strategies for the Prevention and Treatment of Autoimmune Disease, Nat. Rev. Immunol., 7 (2007) 665-677.
12. Pearson et al., Controlled Delivery of Single or Multiple Antigens in Tolerogenic Nanoparticles Using Peptide-Polymer Bioconjugates, Mol. Ther., 25 (2017) 1655-1664. PMC5498834.

1. A method of presenting antigen in a cell, comprising:
(a) obtaining a peptide or protein antigen;
(b) covalently linking the antigen to a lipid to form a conjugate;
(c) introducing the lipid-antigen conjugate into a nanoparticle; and
(d) loading the cells with antigen by contacting the cells with the nanoparticles,
wherein the lipid is compatible with incorporation into nanoparticles and delivering the antigen into the plasma membrane of cells,
wherein the nanoparticle comprises biomaterials compatible with the lipid and the cell and can deliver the antigen to the cell, and
wherein the antigen-loaded cells produce an antigen-specific T cell response.

2. The method of claim 1, wherein the cells are macrophages, dendritic cells, resting B cells, or activated B cells.

3. The method of claim 1, wherein the antigen is an MHCI antigen.

4. The method of claim 1, wherein the antigen is an MHCII antigen.

5. The method of claim 1, wherein the lipid is selected from the group consisting of 1,2-distearoyl-sn-glycero-3-phosphorylethanolamine (DSPE), DSPE-PEG, stearic acid, stearic acid-PEG, cholesterol, cholesterol-PEG, distearoylphosphatidylcholine (DSPC), and DSPC-PEG.

6. The method of claim 1, wherein the nanoparticle comprises poly(lactic-co-glycolic acid), poly(lactic acid), poly(ε-caprolactone), polystyrene, poly(methyl methacrylate), N-(2-hydroxypropyl)-methacrylamide, poly(ethylene glycol), poly(glycolic acid), polyanhydrides, poly(cyano-acrylates), poly(maleic acid), poly(N-vinyl pyrrolidine), chitosan, hyaluronic acid, albumin, heparin, DSPE, DSPE-PEG, palmitoyl, stearic acid, stearic acid-PEG, cholesterol, cholesterol-PEG, DSPC, DSPC-PEG, or mixtures thereof.

7. The method of claim 6, wherein the nanoparticle comprises poly(lactic-co-glycolic acid), poly(lactic acid), or a mixture thereof.

8. The method of claim 1, wherein the antigen-specific T cell response is immune tolerance.

9. The method of claim 1, wherein the antigen-specific T cell response is immune stimulation.

10. The method of claim 1, wherein the antigen loading is performed ex vivo or in vivo.

11. An antigen presenting B cell produced by the method of claim 1.

12. A nanoparticle comprising an antigen-lipid conjugate,
wherein the antigen is a protein or peptide,
wherein the lipid is compatible with incorporation into nanoparticles and delivering the antigen into the plasma membrane of cells,
wherein the nanoparticle comprises poly(lactic-co-glycolic acid), poly(lactic acid), poly(ε-caprolactone), polystyrene, poly(methyl methacrylate), N-(2-hydroxypropyl)-methacrylamide, poly(ethylene glycol), poly(glycolic acid), polyanhydrides, poly(cyano-acrylates), poly(maleic acid), poly(N-vinyl pyrrolidine), chitosan, hyaluronic acid, albumin, heparin, DSPE, DSPE-PEG, stearic acid, stearic acid-PEG, cholesterol, cholesterol-PEG, DSPC, DSPC-PEG, palmitoyl, or mixtures thereof.

13. The nanoparticle of claim 12, wherein the nanoparticle comprises poly(lactic-co-glycolic acid), poly(lactic acid), or a mixture thereof.

14. An antigen presenting cell which has been loaded with antigen by interaction with the nanoparticle of claim 12.

15. A method of presenting antigen in a resting B cell comprising contacting a resting B cell with the nanoparticle of claim 12.

16. A method of presenting antigen in an activated B cell comprising contacting an activated B cell with the nanoparticle of claim 12.

17. A method of treating autoimmune disease or cancer in a subject in need comprising administering the antigen presenting cell of claim 14.

18. A method of inducing an antigen-specific immune reaction in a subject in need thereof, comprising administering the antigen presenting cell of claim 14 to the subject.

19. The method of claim 18, wherein the subject is suffering from an autoimmune disorder or cancer.

20. The method of claim 18, wherein the autoimmune disease is multiple sclerosis.


SEQUENCE LISTING (5 sequences)

SEQ ID NO: 1 - protein, 8 aa, Artificial Sequence
  Synthetic
SIINFEKL

SEQ ID NO: 2 - protein, 17 aa, Artificial Sequence
  Synthetic
ISQAVHAAHAEINEAGR

SEQ ID NO: 3 - protein, 21 aa, Artificial Sequence
  Synthetic
CAVGALEGPRNQDWLGVPRQL

SEQ ID NO: 4 - protein, 9 aa, Artificial Sequence
  Synthetic
KVPRNQDWL

SEQ ID NO: 5 - protein, 17 aa, Artificial Sequence
  Synthetic
ASFEAQGALANIAVDKA